(19) United States
(12) Patent Application Publication
(10) Pub. No.: US 2011/0044933 A1
(43) Pub. Date: Feb. 24, 2011

(54) PARTICLES, OBTAINED BY DRYING AN AQUEOUS NANOUREA DISPERSION

(75) Inventors: Sebastian Dörr, Dusseldorf (DE); Harald Blum, Hafenlohr (DE); Steffen Hofacker, Odenthal (DE); Sophie Viala, Köln (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/935,980

(22) PCT Filed: Mar. 24, 2009
(86) PCT No.: PCT/EP2009/002131

§ 371 Date: Oct. 1, 2010

(30) Foreign Application Priority Data
Apr. 2, 2008 (EP) 08153931.4

(51) Int. Cl. A61K 8/88; C08G 71/02; C08L 75/02

(52) U.S. Cl. 424/78.37; 528/367; 428/402; 524/610; 977/788; 977/926

(57) ABSTRACT

The present invention relates to particles produced by drying aqueous dispersions of nanoureas and to methods for the production thereof.

The present invention relates to particles which can be prepared by drying aqueous dispersions of nanoureas, and to a corresponding process for their preparation. The present invention further provides their use. In addition, the present invention relates to the use of aqueous dispersions of crosslinked polyureas for the preparation of cosmetics.

The spray-drying of solutions of polyureas in organic solvents is already described in EP 1 630 191 A. In this publication, however, neither is the drying of aqueous dispersions disclosed, nor is water used as solvent. In addition, this publication is focused on polyureas to be dried, which have a linear structure. Cross-branched nanoparticles are not discussed. The polyureas described in EP 1 630 191 A are prepared by polyaddition of amines onto isocyanates, where a build-up reaction through chain extension by means of hydrolysis is not described.

The preparation of aqueous dispersions comprising crosslinked, nanoscale polyurea particles is described in WO 2005/063873 A. In this process, hydrophilic isocyanates are placed in water in the presence of a catalyst, as a result of which crosslinking within the dispersed particles is developed through urea bonds. DE 10 2006 008 69 A also describes the preparation of aqueous dispersions comprising crosslinked urea particles. The particle sizes of the particles are in ranges from 10 to 300 nm (measured via laser correlation spectroscopy). These particles are used as additives for contact adhesives based on polychloroprene dispersions.

Since many applications such as, for example, cosmetics, coating compositions, sealants or adhesives require pulverulent fillers with particle diameters in the range from in general 1 to 50 μm, there is a need for the provision of corresponding powders.

One object of the present invention is therefore the provision of polyurea-containing powders in particle form, where the particles should preferably have an average diameter of from 1 to 50 μm.

According to the invention, it has now been found that powders can be obtained by drying aqueous dispersions of crosslinked nanourea particles.

The present invention therefore provides a nanourea powder which is obtained starting from an aqueous dispersion of crosslinked nanoureas by drying. In this process, it was not foreseeable that through the drying of a corresponding nanourea dispersion, a powder product is obtained, but rather a mass with sticky consistency.

The present invention further provides a process for the preparation of nanourea powders by drying aqueous dispersions of polyureas.

The present invention likewise provides the use of the particles which are prepared by drying aqueous dispersions of nanoureas in cosmetics, coating compositions, sealants or adhesives.

The present invention likewise provides the use of the particles which have been prepared by drying aqueous dispersions of nanoureas as filler, additive, auxiliary and/or supplement.

The present invention likewise provides cosmetics, articles, coating compositions, sealants and adhesives which are obtained using the particles according to the invention.

Within the context of the present invention, nanoureas are to be understood as meaning cross-branched polyurea particles with a diameter in the nanoscale range.

Within the context of the present invention, in one embodiment, a nanourea powder which is obtained by freeze-drying an aqueous nanourea dispersion is excluded.

Within the context of the present invention, a further embodiment excludes a nanourea powder which is obtained by freeze-drying an aqueous nanourea dispersion which is obtained in such a way that 820.20 g of Bayhydur® VP LS 2336 and then 0.32 g of Isofoam® 16 are added to a solution of 20.72 g of triethylamine in 4952 g of deionized water at 30° C. with vigorous stirring and the mixture is further stirred, after 3, 6 and 9 hours a further 820.20 g of Bayhydur® VP LS 2336 and then 0.32 g of Isofoam® 16 are added in each case and then the mixture is afterstirred at 30° C. for a further 4 hours and is stirred at 200 mbar vacuum and 30° C. for a further 3 hours and the resulting dispersion is drawn off.

The polyurea powders according to the invention are preferably powders which are obtained starting from aqueous dispersions of crosslinked nanourea particles.

The average particle diameters of the particles according to the invention (determined through measurement by means of optical spectroscopy through adjustment of the found particle diameters using a calibrated longitudinal scale) have sizes of in general from 0.5 to 1000 μm, preferably from 1 to 200 μm, particularly preferably from 1 to 50 μm.

The residual water content of the particles according to the invention which are obtained by drying the nanourea dispersion and which is determined by gravimetric analysis upon further drying of a sample of ca. 1 g of the powder in a convection oven at 120° C. to constant weight, is generally below 10% by weight, preferably from 0.001 to 5% by weight, particularly preferably from 0.1 to 3% by weight.

The content of nanourea particles according to the invention in dispersions in articles according to the invention, in particular cosmetics, coating compositions, sealants or adhesives, is generally from 0.01 to 50% by weight, preferably from 0.1 to 20% by weight.

The particles according to the invention are obtained by drying aqueous nanourea dispersions. In this process, the nanourea particles in the aqueous dispersion are intraparticulately crosslinked essentially through urea bonds. According to the invention, the term “crosslinked essentially through urea bonds” is understood as meaning when preferably at least 50 mol % of the branching sites, preferably at least 90 mol %, in each case starting from the isocyanate-containing hydrophilic starting building block, are crosslinked.

Corresponding nanourea dispersions are obtainable, for example, according to WO 2005/063873 A1, the disclosure of which in this regard is incorporated into the present invention by reference.

The uncrosslinked or precrosslinked particles are formed through dispersion of hydrophilized polyisocyanates i) in water. Then, some of the isocyanate groups present are broken down by an isocyanate/water reaction to give the primary or secondary amine. By reacting with further isocyanate groups, these amino groups then form urea groups and crosslink as a result to give nanourea particles which are present in aqueous nonourea dispersion. Some of the isocyanate groups here can also be reacted with water or with other isocyanate-reactive species, such as, for example, primary or secondary amines and/or alcohols, before or during the reaction.

Hydrophilized polyisocyanates i) which can be used are per se all NCO-group-containing compounds known to the person skilled in the art that have been nonionically or potentially ionically hydrophilized. If mixtures of different polyisocyanates i) are used, it is preferred for at least one polyisocyanate to have a nonionically hydrophilizing structural unit. Exclusively polyisocyanates i) with nonionically hydrophilizing groups are particularly preferably used.

Ionically or potentially ionically hydrophilizing compounds are understood as meaning all compounds which have at least one isocyanate-reactive group and at least one functionality, such as, for example, —COOY, —SO₃Y, —PO(OY)₂ (Y for example ═H, NH₄⁺, metal cation), —NR₂, —NR₃⁺ (R═H, alkyl, aryl), which, upon interaction with aqueous media, enters into a pH-dependent dissociation equilibrium and in this way may carry a negative, positive or neutral charge. Preferred isocyanate-reactive groups are hydroxyl or amino groups.

Suitable ionically or potentially ionically hydrophilizing compounds are, for example, mono- and dihydroxycarboxylic acids, mono- and diaminocarboxylic acids, mono- and dihydroxysulphonic acids, mono- and diaminosulphonic acids, and also mono- and dihydroxyphosphonic acids or mono- and diaminophosphonic acids and their salts, such as dimethylolpropionic acid, dimethylolbutyric acid, hydroxypivalic acid, N-(2-aminoethyl)-β-alanine, 2-(2-aminoethylamino)ethanesulphonic acid, ethylenediaminepropyl- or -butylsulphonic acid, 1,2- or 1,3-propylenediamine-β-ethylsulphonic acid, malic acid, citric acid, glycolic acid, lactic acid, glycine, alanine, taurine, lysine, 3,5-diaminobenzoic acid, an addition product of IPDI and acrylic acid (EP-A 0 916 647, Example 1) and its alkali metal and/or ammonium salts; the adduct of sodium bisulphite onto butene-2-diol-1,4, polyether sulphonate, the propoxylated adduct of 2-butenediol and NaHSO₃, described for example in DE-A 2 446 440 (pages 5-9, formula and also compounds which contain building blocks which can be converted into cationic groups, e.g. amine-based building blocks, such as N-methyldiethanolamine as hydrophilic synthesis components. Furthermore, cyclohexylaminopropanesulphonic acid (CAPS) as for example in WO 01/88006 A can be used as compound.

Preferred ionic or potentially ionic compounds are those which have carboxy or carboxylate and/or sulphonate groups and/or ammonium groups. Particularly preferred ionic compounds are those which contain carboxyl and/or sulphonate groups as ionic or potentially ionic groups, such as the salts of N-(2-aminoethyl)-β-alanine, of 2-(2-aminoethylamino)ethanesulphonic acid or of the addition product of IPDI and acrylic acid (EP 0 916 647 A, Example 1), and also of dimethylolpropionic acid.

Suitable nonionically hydrophilizing compounds are, for example, polyoxyalkylene ethers which contain at least one hydroxy or amino group. These polyethers contain a fraction of from 30% by weight to 100% by weight of building blocks derived from ethylene oxide.

Hydrophilic synthesis components for incorporating terminal hydrophilic chains having ethylene oxide units are preferably compounds of the formula (I),

H—Y′—X—Y—R (I)

in which
R is a monovalent hydrocarbon radical having 1 to 12 carbon atoms, preferably an unsubstituted alkyl radical having 1 to 4 carbon atoms;
X is a polyalkylene oxide chain having 5 to 90, preferably 20 to 70, chain members which consist, to an extent of at least 40%, preferably at least 65%, of ethylene oxide units and which besides ethylene oxide units can consist of propylene oxide, butylene oxide or styrene oxide units, the latter units preferably being propylene oxide units, and
Y′/Y is oxygen or —NR′—, where R′ corresponds with regard to its definition to R or hydrogen.

Particular preference is given to the mixed polymers of ethylene oxide with propylene oxide having an ethylene oxide mass fraction greater than 50%, particularly preferably from 55 to 89%. In one preferred embodiment compounds with a molecular weight of at least 400 g/mol, preferably of at least 500 g/mol and particularly preferably from 1200 to 4500 g/mol are used.

Particular preference is given to nonionically hydrophilized polyisocyanates i) which have, on statistical average, 5 to 70, preferably 7 to 55, oxyethylene groups, preferably ethylene groups, per molecule.

The hydrophilized polyisocyanates i) are based on the aliphatic, cycloaliphatic, araliphatic and aromatic polyisocyanates that are known per se to the person skilled in the art and that have more than one NCO group per molecule and an isocyanate content of from 0.5 to 50% by weight, preferably 3 to 30% by weight, particularly preferably 5 to 25% by weight, or mixtures thereof.

Examples of suitable polyisocyanates are butylene diisocyanate, tetramethylene diisocyanate, cyclohexane-1,3- and 1,4-diisocyanate, hexamethylene diisocyanate (HDI), 1-isocyanato-3,3,5-trimethyl-5-isocyanatomethylcyclohexane (isophorone diisocyanate, IPDI), 2,4,4-trimethylhexamethylene diisocyanate, isocyanatomethyl-1,8-octane diisocyanate methylenebis(4-isocyanatocyclohexane), tetramethylxylylene diisocyanate (TMXDI) or triisocyanatononane (TIN, 4-isocyanatomethyl-1,8-octanediisocyanate) and mixtures thereof. Also suitable in principle are aromatic polyisocyanates such as 1,4-phenylene diisocyanate, 2,4- and/or 2,6-toluene diisocyanate (TDI), diphenylmethane-2,4′- and/or 4,4′-diisocyanate (MDI), triphenylmethane-4,4′-diisocyanate or naphthylene-1,5-diisocyanate.

Besides the aforementioned polyisocyanates, it is also possible to use higher molecular weight secondary products with a uretdione, isocyanurate, urethane, allophanate, biuret, iminooxadiazinedione and/or oxadiazinetrione structure. Such secondary products are known in a manner known per se from the monomeric diisocyanates through the modifying reactions described in the prior art.

Preferably, the hydrophilized polyisocyanates i) are based on polyisocyanates or polyisocyanate mixtures of the type specified above with exclusively aliphatically or cycloaliphatically bonded isocyanate groups or any desired mixtures thereof.

The hydrophilized polyisocyanates are particularly preferably based on hexamethylene diisocyanate, isophorone diisocyanate or the isomeric bis(4,4′-isocyanatocyclohexyl)methanes and also mixtures of the aforementioned diisocyanates. The polyisocyanates i) preferably contain at least 50% by weight of polyisocyanates based on hexamethylene diisocyanate.

The dispersing of the polyisocyanates a) in water and reaction with water for the preparation of the aqueous dispersion preferably takes place with intermixing by means of a stirrer or other types of intermixing, such as circulation pumping, static mixer, barbed mixer, nozzle jet disperser, rotor and stator, or under the influence of ultrasound.

In principle, it is also possible, during or after the dispersing, for NCO groups to be modified with isocyanate-reactive compounds such as primary or secondary amines or (poly) alcohols. Examples thereof are ethylenediamine, 1,3-propylenediamine, 1,6-hexamethylenediamine, isophoronediamine, 4,4′-diaminodicyclohexylmethane, hydrazine, 1,4-butanediol, 1,4-cyclohexanedimethanol, 1,6-hexanediol, trimethylolethane, trimethylolpropane, glycerol, N-methylethanolamine and N-methylisopropanolamine, 1-aminopropanol or diethanolamine.

The molecular ratio of NCO groups of the hydrophilized polyisocyanate i) to water is preferably 1:100 to 1:5, particularly preferably 1:30 to 1:10. Observing this ratio is advantageous to be able to obtain a stable dispersion and to dissipate the reaction enthalpy.

In principle, it is possible to incorporate the hydrophilized polyisocyanate i) into the water in one portion by dispersion. A continuous addition of the hydrophilized polyisocyanate, for example over a period of from 30 minutes to 20 hours, is likewise possible. Preference is given to addition in portions, where the number of portions is 2 to 50, preferably 3 to 20, particularly preferably 4 to 10, and the portions can be identical or different in size.

The waiting time between the individual portions is typically 5 minutes to 12 hours, preferably 10 minutes to 8 hours, particularly preferably 30 minutes to 5 hours.

Continuous addition of the hydrophilized polyisocyanate i) spread over a period of from 1 hour to 24 hours, preferably 2 hours to 15 hours, is likewise possible.

During the urea particle preparation, the temperature in the reactor is 10 to 80° C., preferably 20 to 70° C. and particularly preferably 25 to 50° C.

Following the reaction of the hydrophilized polyisocyanate i) with water, the reactor is preferably evacuated at internal temperatures of from 0 to 80° C., preferably 20 to 60° C., particularly preferably 25 to 50° C. Evacuation takes place down to an internal pressure of from 1 to 900 mbar, preferably from 10 to 800 mbar, particularly preferably 100 to 400 mbar. The duration of this degassing, which follows the actual reaction, is 1 minute to 24 hours, preferably 10 minutes to 8 hours. Degassing is also possible through temperature increase without evacuation.

Preferably, the nanourea dispersion A′) is thoroughly mixed at the same time as the evacuation, e.g. by stirring.

The preparation of the aqueous dispersions A′) preferably takes place in the presence of catalysts.

The catalysts used for the preparation of the nanourea dispersions A′) are, for example, tertiary amines, tin compounds, zinc compounds or bismuth compounds or basic salts.

Suitable catalysts are, for example, iron(II) chloride, zinc chloride, tin salts, tetraalkyl-ammonium hydroxides, alkali metal hydroxides, alkali metal alkoxides, alkali metal salts of long-chain fatty acids having 10 to 20 carbon atoms and optionally lateral OH groups, lead octoate or tertiary amines such as triethylamine, tributylamine, dimethylbenzylamine, dicyclohexylmethylamine, dimethylcyclohexylamine, N,N,N′,N′-tetramethyldiaminodiethyl ether, bis(dimethylaminopropyl)urea, N-methyl- or N-ethylmorpholine, N,N′-dimorpholinodiethyl ether (DMDEE), N-cyclohexylmorpholine, N,N,N′,N′-tetramethylethylenediamine, N,N,N′,N′-tetramethylbutanediamine, N,N,N′,N′-tetramethylhexanediamine-1,6, pentamethyldiethylenetriamine, dimethylpiperazine, N-dimethylaminoethylpiperidine, 1,2-dimethylimidazole, N-hydroxypropylimidazole, 1-azabicyclo(2.2.0)octane, 1,4-diazabicyclo(2.2.2)octane (Dabco) or alkanolamine compounds, such as triethanolamine, triisopropanolamine, N-methyl- and N-ethyldiethanolamine, dimethylaminoethanol, 2-(N,N-dimethylaminoethoxy)ethanol or N-tris(dialkylaminoalkyl)hexahydrotriazines, e.g. N,N′,N-tris(dimethylaminopropyl)-s-hexahydrotriazine.

Preference is given to tertiary amines such as tributylamine, triethylamine, ethyldiisopropylamine or 1,4-diazabicyclo[2.2.2]octane. Preferred tin compounds are tin dioctoate, tin diethylhexoate, dibutyltin dilaurate or dibutyldilauryltin mercaptide. Preference is additionally given to 2,3-dimethyl-3,4,5,6-tetrahydropyrimidine, tetramethylammonium hydroxide, sodium hydroxide, sodium methoxide or potassium isopropoxide.

Particularly preferred catalysts are sodium hydroxide, triethylamine, ethyldiisopropylamine or 1,4-diazabicyclo[2.2.2]octane.

The catalysts are used in amounts of preferably from 0 to 8% by weight, preferably from 0.05 to 5% by weight, particularly preferably from 0.1 to 3% by weight, in each case based on the total solids content of the resulting dispersion.

The catalyst can be mixed with the hydrophilized polyisocyanates i) or with the dispersing water, or can be added after the polyisocyanates i) have been dispersed in water. It is preferred to admix the catalyst to the dispersing water prior to the addition of the polyisocyanate i). It is also possible to divide the catalyst into portions and to add them at different points during the course of the reaction.

It is likewise possible to add solvents such as N-methylpyrrolidone, N-ethylpyrrolidone, methoxypropyl acetate, dimethyl sulphoxide, methyoxypropyl acetate, acetone and/or methyl ethyl ketone to the hydrophilized polyisocyanate i) prior to the dispersing. When the reaction and dispersing are complete, volatile solvents such as acetone and/or methyl ethyl ketone can be removed by distillation. Preference is given to the preparation without solvent or the use of acetone or methyl ethyl ketone, particular preference is given to the preparation without organic solvent.

In principle, the removal of the water is possible at atmospheric pressure, subatmospheric pressure or superatmospheric pressure. In one preferred process variant, the water is removed by distillation, it being possible to operate under reduced pressure and/or elevated temperature.

Other techniques for separating off water are also possible, such as, for example, dewatering by membrane methods or the use of water-removing drying agents, such as, for example, silica gel or zeolites. The combination of different dewatering techniques, simultaneously or in succession, is also possible. Separating off the water with the aid of additives is also possible, for example the admixing of entrainers for the simplified distillative removal of water.

Particular preference is given to drying by means of freeze-drying methods or spray-drying, very particular preference being given to spray-drying.

The spray-drying can be carried out with the help of customary methods. For example, processing can be with the aid of a rotary atomizer, a pressure atomizer or by means of pneumatic atomization. Preference, however, is given to operating using a pressure atomizer.

For the spray-drying, in particular a heated gas, in particular a heated inert gas, preferably heated air or heated nitrogen, are used for the drying. The gas is preferably heated to a temperature such that at the respective discharge rate through the nozzle, the temperature at the spray particle is so high that on the one hand good vaporization of the water or solvent is ensured and on the other hand the temperature on the spray particle is at most so high that no agglutinations/crosslinkings or the like arise. The temperature at the spray particle should particularly preferably be at most 80° C., very particularly preferably 30 to 70° C. Gas which has been heated to a temperature of from 50 to 170° C. is particularly preferably used.

The amount of heat required for vaporizing the water can, however, of course also be partly or completely introduced by other methods, for example radiation heating.

The dispersion to be sprayed used for the spray-drying preferably has a solids content of from 5 to 60% by weight, particularly preferably from 20 to 50% by weight.

The spraying of the particles can take place in particular by means of a rotary atomizer, by means of a pressure atomizer or by means of a pneumatic atomizer, preferably by means of a rotary atomizer. The use of a rotary atomizer here has the advantage that very finely divided powders with a narrow particle size distribution are obtained. Furthermore, particularly in the case of spray-drying using a rotary atomizer, the resulting relatively large powder particles are primarily aggregates of smaller particles.

If appropriate, the spray-drying can be followed by a sifting operation (in particular by means of a wind sifter).

In the case of the use of a rotary atomizer, the liquid pressure is generally 2 to 3 bar. In the case of the use of a pneumatic atomizer or a pressure atomizer, the liquid pressure is preferably 20 to 50 bar.

In the case of the rotary atomizer, the diameter of the plant is preferably between 2.5 and 3.5 m and the length is preferably 4.5 to 5.5 m. The temperature of the gas used for the drying is within the limits given above, preferably 100 to 200° C. The amount of drying gas, preferably drying air, is preferably 1000 to 5500 cubic metres (STP) per hour.

In the case of pressure atomization and pneumatic atomization, processing is likewise with customary plants using inert gases or air. In the case of pneumatic atomization, the amount of atomization air or gas used per tonne of material to be atomized is generally about 1 tonne and processing is preferably carried out using fine nozzles.

The throughflow rate through the nozzle during the spray-drying is preferably adjusted so that, for example on a plant from Niro Atomizer 50 ml to 800 ml are sprayed per minute.

The powders prepared by means of spray-drying generally have an average particle size between 5 and 50 μm. If the powders are spray-dried using a rotary atomizer, then the average particle size is generally below 15 μm. When using a pneumatic atomizer, the average particle size is generally below 50 μm and when using a pressure atomizer is generally 20 to 80 μm. The average particle size can be influenced, for example, by the concentration of the dispersion used, the nozzle diameters, the nozzle geometry, the throughflow rate through the nozzle or temperature and flow rate of the drying gas, it being possible for the person skilled in the art to establish the particle size through routine experiments by varying the above parameters.

If a narrow particle size distribution of the powders is desired, the spray-drying can also be followed by a sifting, in particular by means of a wind sifter. By means of this it is possible to adjust the average particle size to, for example, 5 to 25 μm, preferably 8 to 20 μm. The removal of possibly undesired coarse or fine fractions is also possible. After the drying, the resulting powder can be further ground.

Examples of spray driers which can be used are instruments from Niro (Denmark), Anhydro (Denmark), Nubilosa, Caldyn, Büchi, APV, Trema etc.

During the preparation of the particles according to the invention it is also possible to use cosolvents, antifoams, surface-active detergents and other auxiliaries and additives. Also the admixing of other dispersed nanoparticles is possible, such as, for example, aqueous colloidally dispersed solution of silicon dioxide. If volatile cosolvents are used, these can be removed again from the nanourea dispersion A) according to the invention, for example together with the removal of the water. It is preferred to work without cosolvents.

The addition of release agents such as, for example, talc is also possible. The addition can take place before, during or after drying the nanourea dispersion.

It is also possible to add dyes, aroma substances, pigments and active ingredients.

Further additives which can be added to the starting dispersion are, for example, catalysts, film-forming polymers, stabilizers, photoprotective agents, antioxidants, biocides, pigments and/or fillers. The addition can take place before, during or after the preparation of the nanourea dispersion.

The particles according to the invention can be used as such for example as additive, binder or auxiliary or additive, for example for coating compositions, surface coatings, paints, adhesives, laminating materials, sealants, printing inks, inks, colorants, dyes, etching agents, corrosion inhibitors and rust inhibitors, impregnating agents, lubricants, slip agents, release agents or coolants, softeners, flow agents, reactive thinners, additives; in cosmetics or as cosmetic raw material, for the production of pharmaceutical formulations, in oils, in sun protection compositions, in or as thickeners, cleaners and pretreatments and in foods of all types.

The particles according to the invention can be introduced, for example, through incorporation using a stirrer or other types of introduction such as circulation pumping, static mixer, barbed mixer, nozzle-jet disperser, rotor and stator, incorporation in the extruder, in the three-roll apparatus or under the influence of ultrasound.

Preferred examples for use of the particles according to the invention are incorporation into cosmetics, coating compositions, sealants or adhesives.

A particularly preferred application of the nanourea powders according to the invention is described below. This application relates to their use in cosmetics.

When selecting a cosmetic product, consumers pay particular attention to the skin feel of the cosmetic composition as it is being applied and after the product has soaked in. Numerous cosmetic products leave behind a greasy sticky skin feel upon application to skin and hair. In particular, in the case of skin cosmetic products, the use of hydrophilic humectants, such as glycerine and lipids as protection barrier substances (such as, for example, mineral oils or polar oils) to combat water loss, leads to the formation of a greasy and sometimes sticky film.

The present invention therefore would also like to provide a cosmetic composition which has smooth properties while being spread on the skin or the hair. Within the context of the present invention, “smooth properties” is essentially to be understood as meaning that, upon application to the skin or the hair, the cosmetic composition has a nongreasy, powdery skin feel, preferably a nonsticky skin feel.

Decorative cosmetic compositions consist, depending on the type of formulation, of up to 80% by weight of dyes, in particular pigments, based on the total weight of the composition. The high fraction of dyes and/or pigments has the disadvantage of leaving behind a rough skin feel upon application. Additionally, on account of the composition being difficult to distribute upon spreading on the skin, numerous decorative cosmetic compositions from the prior art lead to a colourwise inhomogeneous make-up product with visible traces of colour.

In general, there is therefore a need for improvement for decorative cosmetic compositions. The development of a decorative cosmetic composition which, upon application to the skin, forms an easily distributable, colourwise homogeneous make-up without visible colour traces is desired. The other important properties of decorative cosmetic products such as, for example, long lasting, should at the same time not be disregarded in the process.

According to the invention, this object is achieved through the use of the polyurea powder according to the invention, which has been described above, and/or a non-film-forming polyurea dispersion comprising crosslinked nanoureas. The non-film-forming polyurea dispersion which can be used is any polyurea dispersion from which, through drying, the polyurea powders according to the invention and described above are produced.

### Cosmetic Composition

The cosmetic composition according to the invention comprising the nanourea powder according to the invention and/or the non-film-forming polyurea dispersion is in the form of aqueous or aqueous-alcoholic solutions, oil-in-water, silicone-in-water, water-in-oil or water-in silicone emulsion and mixed form, multiple emulsion, such as, for example, oil-in-water-in-oil, water-in-oil-in-water emulsion, polymer-stabilized emulsion (so-called hydrodispersion), solids-stabilized emulsion (also called Pickering emulsion), PIT formulation and powder in the form of creams, lotions, foams, sprays (pump spray or aerosol), gels, gel sprays, oils, oil gels, mousse, loose powder, compact powder or stick formulations for use on hair and/or skin.

The cosmetic compositions according to the invention comprise the nanourea powders according to the invention and/or the non-film-forming polyurea dispersion and also optionally active ingredients and auxiliaries customary in cosmetics which are selected from the group consisting of emulsifiers; surfactants; preservatives; perfume oils; cosmetic active ingredients, such as phytantriol, vitamin A, E and C, retinol, bisabolol, panthenol; organic and inorganic photoprotective agents; bleaches, colourants, tints, tanning agents, stabilizers; pH regulators; dyes; salts; thickeners; gel formers; consistency regulators; silicones; humectants; conditioning agents; film formers; refitting agents and further customary additives.

The cosmetic compositions according to the invention can, inter alfa, be formulated as

(1) skin care composition or
(2) hair cosmetic composition,
although the present invention is not limited to these specific types of cosmetic compositions.

### Skin Care Composition

Within the context of the invention, the cosmetic composition can be a skin cosmetic composition. A skin cosmetic composition is defined as a cosmetic composition for the cleansing, care and protection of the skin. Within the context of the present invention, skin cosmetic compositions are skin care product, sunscreen composition, aftersun preparations, self-tanning compositions, decorative cosmetic, washing, showering and bathing preparations for use on the skin, face toners, face masks, insect repellent preparations, footcare compositions, shaving compositions, hair removal compositions, intimate care compositions, babycare compositions, deodorants and antiperspirants.

Preferred skin cosmetic compositions within the context of the present invention are skin care products, sunscreen compositions, self-tanning compositions, and decorative cosmetics.

A skin care product is a cosmetic composition for application to the skin, the face and/or the body to protect against changes in the skin, for example skin ageing, drying etc.

Depending on their formula, the compositions according to the invention can be used, for example, as face cream, day or night cream, eye cream, antiwrinkle cream, whitening products, body lotion, impregnation medium, after-sun preparations etc. It is in some cases possible for the compositions according to the invention to be used as pharmaceutical product.

Within the context of the present invention, insect repellent preparations are preparations which are used externally for protection against and repelling of insects, in particular of flies, ticks and mites. In such formulations, active ingredients are used which keep the insects distant from the skin on account of the formation of a scent mantle above the skin.

A sunscreen composition is a composition to protect the skin against short-wave and long-wave solar radiation. A sunscreen composition comprises at least one photoprotective filter substance (UVA, UVB and/or broadband filters).

The compositions according to the invention can be formulated as skin care products comprising the nanourea powder according to the invention and/or the non-film-forming polyurea dispersion, humectants and optionally further cosmetic and/or dermatological active ingredients, auxiliaries and supplements.

The compositions according to the invention can be formulated as insect repellent preparations comprising at least one nanourea powder according to the invention and/or the non-film-forming polyurea dispersion, insect repellent active ingredients and optionally further cosmetic and/or dermatological active ingredients, auxiliaries and supplements.

The insect repellent active ingredients used are advantageously ethyl 3-(N-n-butyl-N-acetylamino)propionate (available under the tradename Repellent 3535), N,N-diethyl-m-toluamide (so-called DEET) and 2-butyl 2-(2-hydroxyethyl)piperidine-1-carboxylate (available under the tradename Bayrepel®).

The compositions according to the invention can also be formulated as sunscreen compositions comprising at least one nanourea powder according to the invention and/or the non-film-forming polyurea dispersion, at least one or more photoprotective filter substances and optionally further cosmetic and/or dermatological active ingredients, auxiliaries and supplements.

Photoprotective filter substances can be selected from the group consisting of UVA, UVB, broadband filters and mixtures thereof.

The compositions according to the invention can be formulated as self-tanning compositions comprising at least one nanourea powder according to the invention and/or the non-film-forming polyurea dispersion, at least one or more self-tanning substances and optionally further cosmetic and/or dermatological active ingredients, auxiliaries and supplements.

Sun-tanning compositions comprise at least one or more self-tanning substances which are preferably selected from the group consisting of glycerol aldehyde, hydroxymethylglyoxal, γ-dialdehyde, erythrulose, 5-hydroxy-1,4-naphthoquinone, 2-hydroxy-1,4-naphthoquinone, 1-, 3-dihydroxyacetone (DHA), 6-aldo-D-fructose and ninhydrin.

A decorative cosmetic formulation is a cosmetic composition for the colourwise freshening of the human skin, the mucosa, semimucosa, the hair and the nails. The decorative formulation according to the invention can be a face make-up (foundation), a tinted (day) cream, a blusher, a rouge, a mascara, an eye liner, a kohl pencil, an eye shadow, a lipstick, a lip gloss for changing the colour or for making-up the body to combat rings under the eyes, inhomogeneous complexion or further imperfections of the skin such as redness, blotches, wrinkles or pimples. The list of decorative products is of course not intended to be limiting within the context of the present invention.

The compositions according to the invention can be formulated as decorative cosmetic compositions comprising at least one nanourea powder according to the invention and/or the non-film-forming polyurea dispersion, at least one dye and optionally further cosmetic and/or dermatological active ingredients, auxiliaries and supplements. The dyes can be selected from the group consisting of soluble dyes; inorganic pigments, such as, for example, iron oxides and chromium oxides; ultramarine; manganese violet; organic pigments and mother of pearl.

The skin cosmetic compositions according to the invention may be solid (stick), liquid (lotion, care oil) or semisolid (cream, ointment or gel-like products). The compositions can be present, for example, in the form of an oil-in-water emulsion, silicone-in-water emulsion, water-in-oil emulsion, water-in-silicone emulsion, oil-in-water-in-oil emulsion or water-in-oil-in-water emulsion. The compositions can also be foamed with a propellant gas (so-called mousse). The aforementioned emulsions can be stabilized by O/W, W/O or W/Si emulsifier, thickener (as for example in the case of a hydrodispersion) or solids (such as, for example, Pickering emulsion). The formulation according to the invention can be present in the form of loose powder or compact powder.

### Hair Cosmetic Composition

A preferred use within the context of the present invention is the use of the powders according to the invention and/or of the non-film-forming polyurea dispersion in a hair cosmetic composition selected from the group consisting of neutralizers for permanent waves, curl relaxers, styling wrap lotion, hair setting composition, hair shaping composition, hair colourant, hair treatments and shampoo.

The hair cosmetic compositions according to the invention comprise at least one powder according to the invention and/or the non-film-forming polyurea dispersion, at least one component which is selected from the group consisting of conditioners, film formers and surfactants, and optionally further cosmetic and/or dermatological active ingredients, auxiliaries and supplement.

### Ingredients for the Cosmetic Compositions According to the Invention

### Oils, Fats, Waxes

The skin and hair cosmetic compositions according to the invention preferably comprise further non-volatile and/or volatile oils, fats and/or waxes.

Nonvolatile oils and fats are advantageously selected from the group consisting of oils and fats of mineral, animal, vegetable or synthetic origin; polar or nonpolar oils and mixtures thereof. Nonvolatile oils and fats of the compositions according to the invention can advantageously be selected from the following substance group:

mineral oils, polar oils, such as triglycerides of capric acid or of caprylic acid, also natural oils such as, for example castor oil;
fats, natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids;
alkyl benzoates; silicone oils such as dimethylpolysiloxanes, diethylpolysiloxanes, diphenylpolysiloxanes and mixed forms thereof.

The polar oils are advantageously selected from the group consisting of:
a) Esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms.
b) Esters of aromatic carboxylic acids and saturated and/or unsaturated, branched and/or unbranched alcohols of chain length from 3 to 30 carbon atoms.
   Such ester oils can then advantageously be selected from the group consisting of:
   isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, isotridecyl isononanoate, 2-ethylhexyl palmitate, 2-ethylhexyl laurate, 2-ethylhexyl isostearate, 2-hexyldecyl stearate, 2-octyldodecyl palmitate, 2-ethylhexyl cocoate, oleyl oleate, oleyl erucate, erucyl oleate, erucyl erucate, dicaprylyl carbonate (Cetiol CC) and cocoglycerides (Myritol 331), and synthetic, semisynthetic and natural mixtures of such esters, e.g. jojoba oil;
c) alkyl benzoates (C12-15-alkyl benzoates (Finsolv® TN from Finetex)) or 2-phenylethyl benzoates (X-Tend 226 from ISP).
d) lecithins and the fatty acid triglycerides, in particular triglycerol esters of saturated and/or unsaturated, branched and/or unbranched alkanecarboxylic acids of chain length from 8 to 24, in particular 12 to 18, carbon atoms.
   For example, the fatty acid triglycerides can be selected from the group consisting of cocoglyceride, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil, apricot kernel oil, avocado oil and the like.
e) dialkyl ethers and dialkyl carbonates, where, for example, dicaprylyl ether (Cetiol® OE from Cognis) and/or dicaprylyl carbonate (for example Cetiol® CC from Cognis) are preferred.
f) saturated or unsaturated, branched or unbranched alcohols, such as, for example, octyldodecanol.

Nonvolatile oils may likewise advantageously also be nonpolar oils which are selected from the group consisting of the branched and unbranched hydrocarbons, in particular mineral oil, Vaseline, paraffin oil, squalane and squalene; polyolefins, for example polydecenes, hydrogenated polyisobutenes, C13-16 isoparaffin and isohexadecane.

Nonpolar nonvolatile oils can be selected from the nonvolatile silicone oils.

Of the nonvolatile silicone oils, the polydimethylsiloxanes (PDMS), which are optionally phenylated, such as phenyltrimethicone, or are optionally substituted by aliphatic and/or aromatic groups or by functional groups, for example hydroxy groups, thiol groups and/or amino groups; polysiloxanes modified with fatty acids, fatty alcohols or polyoxyalkylenes and mixtures thereof can be stated.

Particularly advantageous oils are 2-ethylhexyl isostearate, octyldodecanol, isotridecyl isononanoate, isoeicosan, 2-ethylhexyl cocoate, C12-15 alkyl benzoate, caprylic/capric triglyceride, dicaprylyl ether, mineral oil, dicaprylyl carbonates, cocoglycerides, butylene glycol, dicaprylate/dicaprate, hydrogenated polyisobutenes, cetearyl isononanoates, isodecyl neopentanoates, squalane and C13-16 isoparaffin.

The compositions according to the invention can also comprise a wax.

Within the context of the present specification, a wax is defined as a lipophilic fatty substance which is solid at room temperature (25° C.) and exhibits a reversible solid/liquid change in state at a melting temperature between 30° C. and 200° C. Above the melting point, the wax is of low viscosity and miscible with oils.

The wax is advantageously selected from the groups of natural waxes, such as, for example, cotton wax, carnauba wax, candelilla wax, esparto wax, Japan wax, Montan wax, sugarcane wax, beeswax, wool wax, shellac, microwaxes, ceresine, ozokerite, ouricury wax, cork fibre wax, lignite waxes, berry wax, shea butter or synthetic waxes such as paraffin waxes, polyethylene waxes, waxes prepared by Fischer-Tropsch synthesis, hydrogenated oils, fatty acid esters and glycerides which are solid at 25° C., silicone waxes and derivatives (alkyl derivatives, alkoxy derivatives and/or esters of polymethylsiloxane) and mixtures thereof. The waxes can be present in the form of stable dispersions of colloidal wax particles which can be prepared by known processes, for example in accordance with “Microemulsions Theory and Practice”, L. M. Prince Ed., Academic Press (1977), pages 21-32.

The compositions according to the invention can also comprise a volatile oil which is selected from the group of volatile hydrocarbon oils, siliconized oils or fluorinated oils.

Within the context of the present invention, a volatile oil is an oil which evaporates in less than one hour upon contact with the skin at room temperature and atmospheric pressure. The volatile oil is liquid at room temperature and has a vapour pressure at room temperature and atmospheric pressure of preferably 0.13 to 40 000 Pa (10⁻³ to 300 mg of Hg), in particular 1.3 to 13 000 Pa (0.01 to 100 mm of Hg), particularly preferably 1.3 to 1300 Pa (0.01 to 10 mm of Hg), and a boiling point of preferably from 150 to 260° C., particularly preferably 170 to 250° C.

A hydrocarbon oil is understood as meaning an oil which is formed essentially from carbon atoms and hydrogen atoms and optionally oxygen atoms or nitrogen atoms and contains no silicon atoms or fluorine atoms, it also being possible for it to consist of carbon atoms and hydrogen atoms; it can contain at least one ester group, ether group, amino group and/or amide group.

A siliconized oil is understood as meaning an oil which contains at least one silicon atom and in particular Si—O groups.

A fluorinated oil is to be understood as meaning an oil which comprises at least one fluorine atom.

The volatile hydrocarbon oil can be selected from the hydrocarbon oils with a flashpoint of generally 40 to 102° C., preferably 40 to 55° C., particularly preferably 40 to 50° C.

For example, the volatile hydrocarbon oils are volatile hydrocarbon oils having 8 to 16 carbon atoms and mixtures thereof, in particular branched C₈₋₁₆-alkanes, such as the isoalkanes (which are also referred to as isoparaffins) having 8 to 16 carbon atoms, in particular isododecane, isodecane and isohexadecane, and also, for example, the oils which are supplied under the trade names Isopars® or Permethyls®; and the branched C₈₋₁₆ esters, such as isohexylneopentanoate and mixtures thereof.

The volatile hydrocarbon oils such as isododecane, isodecane and isohexadecane are particularly advantageous.

The volatile siliconized oil can be selected among the siliconized oils with a flashpoint of in general 40 to 102° C., preferably a flashpoint above 55° C. and at most 95° C., particularly preferably in the range from 65 to 95° C.

For example, for the volatile siliconized oils, the straight-chain or cyclic silicone oils having 2 to 7 silicon atoms can be mentioned, where these silicones optionally contain alkyl or alkoxy groups having 1 to 10 carbon atoms.

The volatile siliconized oils such as octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof are particularly advantageous.

The volatile fluorinated oil generally has no flashpoint.

For example, the volatile fluorinated oils are nonafluoroethoxybutane, nonafluoromethoxybutane, decafluoropentane, tetradecafluorohexane, dodecafluoropentane and mixtures thereof.

If present, the fatty phase of the compositions according to the invention can comprise a nonvolatile oil and/or volatile oil, fats and waxes. The O/W composition comprises preferably 0.01 to 45% by weight of oils, particularly preferably 0.01 to 20% by weight of oils, in each case based on the total weight of the composition. The W/O or W/Si composition preferably comprises at least 20% by weight of oils, based on the total weight of the composition.

### Thickeners

The compositions according to the invention may, if an aqueous phase is present, advantageously comprise thickeners (of the water phase). Advantageous thickeners are:
homopolymers or copolymers of crosslinkers or noncrosslinked acrylic acid or methacrylic acid. These include crosslinked homopolymers of methacrylic acid or acrylic acid, copolymers of acrylic acid and/or methacrylic acid and monomers which are derived from other acryloyl or vinyl monomers, such as C10-30 alkyl acrylates, C10-30-alkyl methacrylates, vinyl acetate and vinylpyrrolidones;
thickening polymers of natural origin, for example based on cellulose, guar gum, xanthan, scleroglucan, gellan gum, rhamsan and karaya gum, alginates, maltodextrin, starch and its derivatives, carob seed flour, hyaluronic acid, carrageenan;
nonionic, anionic, cationic or amphoteric associative polymers, e.g. based on polyethylene glycols and their derivatives, or polyurethanes; and crosslinked or noncrosslinked homopolymers or copolymers based on acrylamide or methacrylamide, such as homopolymers of 2-acrylamido-2-methylpropanesulphonic acid, copolymers of acrylamide or methacrylamide and methacryloyloxyethyltrimethylammonium chloride or copolymers of acrylamide and 2-acrylamido-2-methylpropanesulphonic acid.

Particularly advantageous thickeners are thickening polymers of natural origin, homopolymers or copolymers of crosslinked acrylic acid or methacrylic acid and crosslinked copolymers of 2-acrylamido-2-methylpropanesulphonic acid.

Very particularly advantageous thickeners are xanthan gum, such as the products supplied under the names Keltrol® and Kelza® by CP Kelco or the products from RHODIA with the name Rhodopol®, and guar gum, such as the products available under the name Jaguar® HP105 from RHODIA.

Very particularly advantageous thickeners are crosslinked homopolymers of methacrylic acid or acrylic acid which are commercially available from Lubrizol under the names Carbopol® 940, Carbopol® 941, Carbopol® 980, Carbopol® 981, Carbopol® ETD 2001, Carbopol® EDT 2050, Carbopol® 2984, Carbopol® 5984 and Carbopol® Ultrez 10; and from 3V under the names Synthalen® K, Synthalen® L and Synthalen® MS.

Very particularly advantageous thickeners are crosslinked copolymers of acrylic acid or methacrylic acid and a C₁₀₋₃₀-alkyl acrylate or C₁₀₋₃₀-alkyl methacrylate and copolymers of acrylic acid or methacrylic acid and vinylpyrrolidones. Such copolymers are commercially available, for example, from Lubrizol under the names Carbopol® 1342, Carbopol® 1382, Pemulen® TR1 or Pemulen® TR2 and from ISP under the names Ultrathix® P-100 (INCI: Acrylic Acid/VP Crosspolymer).

Very particularly advantageous thickeners are crosslinked copolymers of 2-acrylamido-2-methylpropanesulphonic acid. Such copolymers are available, for example, from Clariant under the names Aristoflex® AVC (INCI: Ammonium Acryloyldimethyltaurate/VP Copolymer).

These thickeners are generally present in a concentration of from about 0% to 2% by weight, preferably 0% to 1% by weight, in each case based on the total weight of the composition.

To stabilize the W/O emulsions according to the invention against sedimentation or flocculation of the water droplets, an oil thickener can be used. Oil thickeners may also be used as consistency regulators in oil-containing compositions.

Particularly advantageous oil thickeners are organomodified clays, such as organomodified bentonites (Bentone® 34 from Rheox), organomodified hectorites (Benton® 27 and Bentone® 38 from Rheox) or organomodified montmorillonite, hydrophobic fumed silica, where the silanol groups are substituted by trimethylsiloxy groups (AEROSIL® R812 from Degussa) or by dimethylsiloxy groups or polydimethylsiloxane (AEROSIL® R972, AEROSIL® R974 from Degussa, CAB-O-SIL® TS-610, “CAB-O-SIL® TS-720 from Cabot), magnesium stearate or aluminium stearate, or styrene copolymers, such as, for example, styrene-butadiene-styrene, styrene-isopropene-styrene, styrene-ethylene/butene-styrene or styrene-ethylene/propene-styrene.

The thickener for the fatty phase can be present in an amount of in general 0.1 to 5% by weight, preferably, 0.4 to 3% by weight, in each case based on the total weight of the composition.

### Emulsifiers

The compositions according to the invention in the form of an emulsion, such as, for example, oil-in-water emulsion, silicone-in-water emulsion, water-in-oil emulsion, water-in-silicone emulsion, oil-in-water-in-oil emulsion, water-in-oil-in-water emulsion may comprise an emulsifier.

As is known by the person skilled in the art, the selection of the emulsifier depends on the application form of the compositions according to the invention. Thus, oil-in-water emulsions (O/W) according to the invention preferably comprise at least one emulsifier with an HLB value of >7 and optionally a coemulsifier. The water-in-oil (W/O) or water-in-silicone (W/Si) emulsions preferably comprise the one or more silicone emulsifiers (W/S) with an HLB value of 8 or one or more W/O emulsifiers with an HLB value of <7 and optionally one or more O/W emulsifiers with an HLB value of >10.

O/W emulsifiers can advantageously be selected from the group of nonionic, anionic, cationic or amphoteric emulsifiers.

The nonionic emulsifiers include:

a) partial fatty acid esters and fatty acid esters of polyhydric alcohols and ethoxylated derivatives thereof;
b) ethoxylated fatty alcohols and fatty acids;
c) ethoxylated fatty amines, fatty acid amides, fatty acid alkanolamides;
d) alkylphenol polyglycol ethers (e.g. Triton X); and
e) ethoxylated fatty alcohol ethers.

Particularly advantageous nonionic O/W emulsifiers are ethoxylated fatty alcohols or fatty acids, preferably PEG-100 stearate, PEG-40 stearate, PEG-50 stearate, ceteareth-20, ceteth-20, steareth-20, ceteareth-12, ceteth-12, steareth-12, esters of mono-, oligo- or polysaccharides with fatty acids, preferably cetearyl glucoside, methylglucose distearate, glyceryl monostearates (self-emulsifying), sorbitan esters, such as, for example, sorbitan stearates (Tween® 20 and Tween® 60 from Uniqema), sorbitan palmitates (span 40, Uniqema), glyceryl stearyl citrates, sucrose esters, such as, for example, sucrose stearates, PEG-20 methylglucose sequistearate), dicarboxylic acid esters of fatty alcohol (dimyristyl tartrates).

Advantageous anionic emulsifiers are soaps (e.g. sodium or triethanolamine salts of stearic acid or palmitic acid), esters of citric acid, such as glyceryl stearate citrate, fatty alcohol sulphates and mono-, di- and trialkylphosphoric acid esters and ethoxylates thereof.

The cationic emulsifiers include quaternary ammonium compounds with a long-chain aliphatic radical, e.g. distearyldimonium chloride.

The amphoteric emulsifiers include:

a) alkylamininoalkanecarboxylic acids;
b) betaines, sulphobetaines; and
c) imidazoline derivatives.

Furthermore, there are naturally occurring emulsifiers, which include beeswax, wool wax, lecithin and sterols.

The silicone emulsifiers can advantageously be selected from the group comprising alkyldimethicone copolyols such as, for example, cetyl PEG/PPG 10/1 dimethicone copolyol (ABIL® EM 90 from Evonik) or lauryl PEG/PPG-18/18 dimethicones (Dow Corning® 5200 Formulation Aid from Dow Corning Ltd.) and dimethicone copolyols such as, for example, PEG-10 dimethicones (KF-6017 from Shin Etsu), PEG/PPG-18/18 dimethicones (Dow Corning Formulation Aid 5225C from Dow Corning Ltd.), PEG/PPG-19/19 dimethicones (Dow Corning BY-11 030 from Dow Corning Ltd.) or trimethylsilylamodimethicones.

The W/O emulsifiers with an HLB value of <7 can advantageously from the group fatty alcohols having 8 to 30 carbon atoms, monoglycerol esters of saturated and/or unsaturated, branched and/or unbranched alkanecarboxylic acids of chain length from 8 to 24, in particular 12-18, carbon atoms, diglycerol esters of saturated and/or unsaturated, branched and/or unbranched alkanecarboxylic acids of chain length from 8 to 24, in particular 12-18, carbon atoms, monoglycerol ethers of saturated and/or unsaturated, branched and/or unbranched alcohols of chain length from 8 to 24, in particular 12-18, carbon atoms, diglyceryl ethers of saturated and/or unsaturated, branched and/or unbranched alcohols of chain length from 8 to 24, in particular 12 to 18, carbon atoms, propylene glycol esters of saturated and/or unsaturated, branched and/or unbranched alkanecarboxylic acids of chain length from 8 to 24, in particular 12 to 18, carbon atoms, and also sorbitan esters of saturated and/or unsaturated, branched and/or unbranched alkanecarboxylic acids of chain length from 8 to 24, in particular 12 to 18, carbon atoms.

Particularly advantageous W/O emulsifiers are: glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprinate and glyceryl monocaprylate.

Further possible W/O emulsifiers are selected from the group of the compounds polyglyceryl-2 dipolyhydroxystearate, PEG-30 dipolyhydroxystearate, cetyl dimethicone copolyol, polyglyceryl-3 diisostearate.

The O/W emulsifiers with an HLB value of >10 can advantageously be selected from the group comprising lecithin, trilaureth-4 phosphate, polysorbate-20, polysorbate-60, PEG-22 dodecyl glycol copolymer, sucrose stearate and sucrose laurate.

Suitable coemulsifiers for the O/W emulsions according to the invention which may be used are fatty alcohols having 8 to 30 carbon atoms, monoglycerol esters of saturated or unsaturated, branched or unbranched alkanecarboxylic acids with a chain length of from 8 to 24 carbon atoms, in particular 12 to 18 carbon atoms, propylene glycol esters of saturated or unsaturated, branched or unbranched alkanecarboxylic acids with a chain length of from 8 to 24 carbon atoms, in particular 12 to 18 carbon atoms, and also sorbitan esters of saturated or unsaturated, branched or unbranched alkanecarboxylic acids with a chain length of from 8 to 24 carbon atoms, in particular 12 to 18 carbon atoms.

Particularly advantageous coemulsifiers are glyceryl monostearate, glyceryl monooleate, diglyceryl monostearate, sorbitan monoisostearate, sucrose distearate, cetyl alcohol, stearyl alcohol, behenyl alcohol, isobehenyl alcohol and polyethylene glycol(2) stearyl ether (steareth-2).

### UV Filters

The compositions according to the invention can comprise sunscreen filters, where the total amount of the sunscreen filters is 0% by weight to 30% by weight, advantageously 0% by weight to 20% by weight, particularly advantageously 0% by weight to 10% by weight, in each case based on the total weight of the composition according to the invention. The sunscreen filters (or UV filters) can be selected from the organic filters, the physical filters and mixtures thereof.

The compositions according to the invention can comprise UV-A filters, UV-B filters or broadband filters. The organic UV filters used may be oil-soluble or water-soluble. The list below of the specified UV filters is of course not limiting.

### Examples of UV-B Filters are:

(1) salicylic acid derivatives, particularly homomethyl salicylate, octyl salicylate and 4-isopropylbenzyl salicylate;
(2) cinnamic acid derivatives, in particular 2-ethylhexyl p-methoxycinnamate, which is available from Givaudan under the name Parsol MCX® and isopentyl 4-methoxycinnamate;
(3) liquid β,β′-diphenyl acrylate derivatives, in particular 2-ethylhexyl α,β′-diphenyl acrylate or octocrylene, which is available from BASF under the name UVINUL N539®;
(4) p-aminobenzoic acid derivatives, in particular 2-ethylhexyl 4-(dimethylamino)benzoate, amyl 4-(dimethylamino)benzoate;
(5) 3-benzylidenecamphor derivatives, in particular 3-(4-methylbenzylidene)camphor which is commercially available from Merck under the name EUSOLEX 6300®, 3-benzylidenecamphor, benzylidenecamphorsulphonic acid and polyacrylamidomethylbenzylidenecamphor;
(6) 2-phenylbenzimidazole-5-sulphonic acid which is available under the name EUSOLEX 232® from Merck;
(7) 1,3,5-triazine derivatives, in particular:
2,4,6-tris[p-(2′-ethylhexyl-1′-oxycarbonyl)anilino]-1,3,5-triazine, which is supplied by BASF under the name UVINUL T150®, and
dioctylbutamidotriazone, which is supplied by Sigma 3V under the name UVASORB HEB®;
(8) esters of benzylmalonic acid, in particular di(2-ethylhexyl) 4-methoxybenzylmalonate and 3-(4-(2,2-bisethoxycarbonylvinyl)phenoxy)propenyl)methoxysiloxane/dimethylsiloxane copolymer, which is available from Roche Vitamins under the name Parsol® SLX; and
(9) the mixtures of these filters.

### Examples of UV-A Filters are:

(1) dibenzoylmethane derivatives, particularly 4-(t-butyl)-4′-methoxydibenzoylmethane, which is supplied by Givaudan under the name PARSOL 1789®, and 1-phenyl-3-(4′-isopropylphenyl)propane-1,3-dione;
(2) benzene-1,4-[di(3-methylidenecamphor-10-sulphonic acid)], optionally completely or partially neutralized, which is commercially available under the name MEXORYL SX® from Chimex;
(3) hexyl 2-(4′-diethylamino-2′-hydroxybenzoyl)benzoate (also aminobenzophenone);
(4) silane derivatives or polyorganosiloxanes with benzophenone groups;
(5) anthranilates, particularly menthyl anthranilate, which is supplied by Symrise under the name NEO HELIOPAN MA®;
(6) compounds which contain at least two benzoazolyl groups or at least one benzodiazolyl group per molecule, in particular 1,4-bisbenzimidazolylphenylene-3,3′,5,5′-tetrasulphonic acid and its salts, which are commercially available from Symrise;
(7) silicon derivatives of benzimidazolylbenzazoles which are N-substituted, or of benzofuranylbenzazoles, in particular:
2-[1-[3-[1,3,3,3-tetramethyl-1-[(trimethylsilyl)oxy]disiloxanyl]propyl]-1H-benzimidazol-2-yl]benzoxazole;
2-[1-[3-[1,3,3,3-tetramethyl-1-[(trimethylsilyl)oxy]disiloxanyl]propyl]-1H-benzimidazol-2-yl]benzothiazole;
2-[1-(3-trimethylsilanylpropyl)-1H-benzimidazol-2-yl]benzoxazole;
6-methoxy-1,1′-bis(3-trimethylsilanylpropyl)1H,1′H-[2,2′]dibenzimidazolylbenzoxazole;
2-[1-(3-trimethylsilanylpropyl)-1H-benzimidazol-2-yl]benzothiazole; which are described in the patent application EP-A-1 028 120;
(8) triazine derivatives, in particular 2,4-bis-[5-1(dimethylpropyl)benzoxazol-2-yl(4-phenyl)imino]-6-(2-ethylhexyl)imino-1,3,5-triazine, which is supplied by 3V under the name Uvasorb K2A; and
(9) mixtures thereof.

### Examples of Broadband Filters are:

(1) benzophenone derivatives, for example
2,4-dihydroxybenzophenone (benzophenone-1);
2,2′,4,4′-tetrahydroxybenzophenone (benzophenone-2);
2-hydroxy-4-methoxybenzophenone (benzophenone-3), which is available from BASF under the name UVINUL M40®;
2-hydroxy-4-methoxybenzophenone-5-sulphonic acid (benzophenone-4), and its sulphonate form (benzophenone-5), which are available from BASF under the name UVINUL MS40®;
2,2′-dihydroxy-4,4′-dimethoxybenzophenone (benzophenone-6);
5-chloro-2-hydroxybenzophenone (benzophenone-7);
2,2′-dihydroxy-4-methoxybenzophenone (benzophenone-8);
the disodium salt of 2,2′-dihydroxy-4,4′-dimethoxybenzophenone-5,5′-disulphonic acid (benzophenone-9);
2-hydroxy-4-methoxy-4′-methylbenzophenone (benzophenone-10);
bis(2,4-dihydroxyphenyl)methanone (benzophenone-11); and
2-hydroxy-4-(octyloxy)benzophenone (benzophenone-12).
(2) triazine derivatives, in particular 2,4-bis{[4-2-ethylhexyloxy)-2-hydroxy]phenyl}-6-(4-methoxyphenyl)-1,3,5-triazine, which is supplied by Ciba Geigy under the name TINOSORB S®, and 2,2′-methylenebis[6-(2H-benzotriazol-2-yl)-4-(1,1,3,3-tetramethylbutyl)phenol], which is available from Ciba Geigy under the name TINOSORB M®; and
(3) 2-(1H-benzotriazol-2-yl)-4-methyl-6-[2-methyl-3-[1,3,3,3-tetramethyl-1-[(trimethylsilyl)oxy]disiloxanyl]propyl]phenol with the INCI name Dromotrizole Trisiloxane.

It is also possible to use a mixture of two or more filters and a mixture of UV-B filters, UV-A filters and broadband filters, and mixtures with physical filters.

Physical filters which may be stated are the sulphate of barium, oxides of titanium (titanium dioxide, amorphous or crystalline in the form of rutile and/or anatase), of zinc, of iron, of zirconium, of cerium, silicon, manganese or mixtures thereof. The metal oxides can be present in particle form with a size in the micrometre range or nanometre range (nanopigments).

The average particle sizes for the nanopigments are, for example, 5 to 100 nm

### Dyes

If appropriate, the compositions according to the invention comprise a dye which is selected from the group of lipophilic dyes, hydrophilic dyes, pigments and mother of pearl. According to the invention, the concentration of dyes is particularly advantageously 0 to 40% by weight, particularly advantageously 0 to 30% by weight, very particularly advantageously from 0 to 25% by weight, in each case based on the total weight of the composition.

For example, the lipophilic dyes can be Sudan I (yellow), Sudan II (orange), Sudan III (red), Sudan IV (scarlet red), DC Red 17, DC Green 6, β-carotene, soybean oil, DC Yellow 11, DC Violet 2, DC Orange 5 and DC Yellow 10.

The pigments may be inorganic or organic pigments which can be used in cosmetic or dermatological composition. The pigments used according to the invention may be white or coloured, and may be coated or not coated with a hydrophobic treatment composition.

The pigments are advantageously selected from the group of metal oxides, such as the oxides of iron (in particular the oxides of yellow, red, brown, black colour), titanium dioxide, zinc oxide, cerium oxide, zirconium oxide, chromium oxide; manganese violet, ultramarine blue, Prussian blue, ultramarine and iron blue, bismuth oxychloride, mother of pearl, mica pigments coated with titanium or bismuth oxychloride, coloured pearlescent pigments, for example titanium-mica pigments with iron oxides, titanium-mica pigments, in particular with iron blue or chromium oxide, titanium-mica pigments with an organic pigment of the aforementioned type, and also pearlescent pigments based on bismuth oxychloride, carbon black, the pigments of the D & C type and the lakes based on cochineal red, barium, strontium, calcium and aluminium and mixtures thereof.

The pigments of iron oxides or titanium dioxide are particularly advantageously used.

For better wettability of the pigments by the oils of the fatty phase, the surface of the pigments can be treated with a hydrophobic treatment composition. The hydrophobic treatment composition is selected from the group of silicones, such as methicones, dimethicones, perfluoroalkylsilanes; fatty acids such as stearic acid; metal soaps, such as aluminium dimyristate, the aluminium salt of hydrogenated tallow glutamate, perfluoroalkyl phosphates, perfluoroalkylsilanes, perfluoroalkylsilazanes, hexafluoropropylene polyoxides, polyorganosiloxanes which contain perfluoroalkyl perfluoropolyether groups, amino acids; N-acylated amino acids or salts thereof; lecithin, isopropyl triisosteaiyltitanate and mixtures thereof. The N-acylated amino acids can contain an acyl group having 8 to 22 carbon atoms, for example 2-ethylhexanoyl, caproyl, lauroyl, myristoyl, palmitoyl, stearoyl or cocoyl. The salts of these compounds may be aluminium salts, magnesium salts, calcium salts, zirconium salts, tin salts, sodium salts or potassium salts. The amino acid may be, for example, lysine, glutamic acid or alanine.

### Conditioning Agents

If appropriate, the compositions according to the invention comprise a conditioning agent. Conditioning agents preferred according to the invention are, for example, all compounds which are listed in the International Cosmetic Ingredient Dictionary and Handbook (Volume 4, editor: R. C. Pepe, J. A. Wenninger, G. N. McEwen, The Cosmetic, Toiletry, and Fragrance Association, 9^{th} edition, 2002) under Section 4 under the keywords Hair Conditioning Agents, Humectants, Skin-Conditioning Agents, SkinConditioning Agents-Emollient, Skin-Conditioning Agents-Humectant, SkinConditioning Agents-Miscellaneous, Skin-Conditioning Agents-Occlusive and Skin Protectants, and also all of the compounds listed in EP-A 934 956 (pp. 11-13) under “water soluble conditioning agent” and “oil soluble conditioning agent”.

Particularly advantageous conditioning substances are, for example, the compounds referred to in accordance with INCI as Polyquaternium (in particular polyquaternium-1 to polyquaternium-56).

Suitable conditioning agents include, for example, also polymeric quaternary ammonium compounds, cationic cellulose derivatives, chitosan derivatives, guar gum derivatives and polysaccharides, in particular guar hydroxypropylammonium chloride (e.g. Jaguar® Excel, Jaguar®162 from Rhodia).

Further conditioning agents advantageous according to the invention are non-ionic poly-N-vinylpyrrolidone/polyvinyl acetate copolymers (e.g. Luviskol® VA 64 from BASF AG), anionic acrylate copolymers (e.g. Luviflex®Soft from BASF AG), and/or amphoteric amide/acrylate/methacrylate copolymers (e.g. Amphomer® from National Starch). Further conditioning agents are quaternized silicones.

### Surfactants

The compositions according to the invention can also comprise surfactants which are selected from the group of anionic, cationic, nonionic and/or amphoteric surfactants.

Advantageous anionic surfactants within the context of the present invention are:
acylamino acids and salts thereof, such as acyl glutamates, in particular sodium acyl glutamate and sarcosinates, for example myristoyl sarcosine, TEA lauroyl sarcosinate, sodium lauroyl sarcosinate and sodium cocoyl sarcosinate;
sulphonic acids and salts thereof, such as acyl isethionates, for example sodium or ammonium cocoyl isethionate, sulphosuccinates, for example dioctyl sodium sulphosuccinate, disodium laureth sulphosuccinate, disodium lauryl sulphosuccinate and disodium undecylenamido MEA sulphosuccinate, disodium PEG-5 lauryl citrate sulphosuccinate and derivatives;
sulphuric acid esters, such as alkyl ether sulphate, for example sodium, ammonium, magnesium, MIPA, TIPA laureth sulphate, sodium myreth sulphate and sodium C12-13 pareth sulphate, and alkyl sulphates, for example sodium, ammonium and TEA lauryl sulphate;
taurates, for example sodium lauroyl taurate and sodium methylcocoyl taurate, ® ether carboxylic acids, for example sodium laureth-13 carboxylate and sodium PEG-6 cocamide carboxylate, sodium PEG-7 olive oil carboxylate;
phosphoric acid esters and salts, such as, for example, DEA oleth-10 phosphate and dilaureth-4 phosphate;
alkylsulphonates, for example sodium coconut monoglyceride sulphate, sodium C12 to C14-olefinsulphonate, sodium lauryl sulphoacetate and magnesium PEG-3 cocamidosulphate;
acyl glutamates, such as di-TEA palmitoyl aspartate and sodium capiylic/capric glutamate,
acyl peptides, for example palmitoyl hydrolysed milk protein, sodium cocoyl hydrolysed soya protein and sodium/potassium cocoyl hydrolysed collagen;
carboxylic acids and derivatives, such as, for example, lauric acid, aluminium stearate, magnesium alkanolate and zinc undecylenate, ester carboxylic acids, for example calcium stearoyl lactylate, laureth-6 citrate and sodium PEG-4 lauramide carboxylate; and
alkylarylsulphonates.

Within the context of the present invention, advantageous cationic surfactants are quaternary surfactants. Quaternary surfactants contain at least one N atom which is covalently bonded to 4 alkyl or aryl groups. Alkylbetaine, alkylamidopropylbetaine and alkylamidopropylhydroxysultaine, for example, are advantageous.

Further advantageous cationic surfactants within the context of the present invention are also alkylamines, alkylimidazoles and ethoxylated amines and in particular salts thereof.

Advantageous amphoteric surfactants within the context of the present invention are acyl/dialkylethylenediamines, for example sodium acyl amphoacetate, disodium acyl amphodipropionate, disodium alkyl amphodiacetate, sodium acyl amphohydroxypropylsulphonate, disodium acyl amphodiacetate, sodium acyl amphopropionate, and N-coconut fatty acid amidoethyl N-hydroxyethylglycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Advantageous active nonionic surfactants within the context of the present invention are alkanolamides, such as cocamides MEA/DEA/MIPA, esters which are formed by esterification of carboxylic acids with ethylene oxide, glyceryl, sorbitan or other alcohols, ethers, for example ethoxylated alcohols, ethoxylated lanoline, ethoxylated polysiloxanes, propoxylated POE ethers, alkyl polyglycosides, such as lauryl glucoside, decyl glycoside and cocoglycoside, glycosides with an HLB value of at least 20 (e.g. Belsil®SPG 128V from Wacker).

Further advantageous nonionic surfactants are alcohols and amine oxides, such as cocoamidopropylamine oxide.

Among the alkyl ether sulphates, preference is given in particular to sodium alkyl ether sulphates based on di- or triethoxylated lauryl and myristyl alcohol. They are significantly superior to the alkyl sulphates with regard to the insensitivity towards water hardness, the ability to be thickened, the solubility at low temperature and in particular the skin and mucosa compatibility. Lauryl ether sulphate has better foam properties than myristyl ether sulphate, but is inferior to this in terms of mildness.

Alkyl ether carboxylates are types of the mildest surfactants in general, but exhibit poor foam and viscosity behaviour. They are often used in combination with alkyl ether sulphates and amphoteric surfactants.

Sulphosuccinic acid esters (sulphosuccinates) are mild and readily foaming surfactants, but on account of their poor ability to be thickened, are preferably used only together with other anionic and amphoteric surfactants and, on account of their low hydrolysis stability, are used preferably only in neutral or well buffered products.

Amidopropylbetaines have excellent skin and eye mucosa compatibility. In combination with other surfactants, their mildness can be improved synergistically. Preference is given to the use of cocamidopropylbetaine.

Amphoacetates/amphodiacetates have, as amphoteric surfactants, very good skin and mucosa compatibility and can have a conditioning effect and/or increase the care effect of supplements. Like the betaines, they are used for optimizing alkyl ether sulphate formulations. Sodium cocoamphoacetate and disodium cocoamphodiacetate are most preferred.

Alkyl polyglycosides are mild, have good universal properties, but are weakly foaming. For this reason, they are preferably used in combinations with anionic surfactants.

### Film Formers

The film former or the film formers are advantageously selected from the group of water-soluble or water-dispersible polyurethanes, the polyureas, silicone resins and/or polyesters, and also the nonionic, anionic, amphoteric and/or cationic polymers and their mixtures.

Advantageous nonionic polymers which may be present in the compositions according to the invention alone or in a mixture, preferably also with anionic and/or amphoteric and/or zwitterionic polymers, are selected from:
polyalkyloxazolines;
vinyl acetate homopolymers or copolymers. These include, for example, copolymers of vinyl acetate and acrylic acid esters, copolymers of vinyl acetate and ethylene, copolymers of vinyl acetate and maleic acid esters;
acrylic acid ester copolymers, such as, for example, the copolymers of alkyl acrylate and alkyl methacrylate, copolymers of alkyl acrylate and urethanes;
copolymers of acrylonitrile and nonionic monomer selected from butadiene and (meth)acrylate;
styrene homopolymers and copolymers. These include, for example, homopolystyrene, copolymers of styrene and alkyl (meth)acrylate, copolymers of styrene, alkyl methacrylate and alkyl acrylate, copolymers of styrene and butadiene, copolymers of styrene, butadiene and vinylpyridine;
polyamides;
vinyllactam homopolymers or copolymers, such as vinylpyrrolidone homopolymers or copolymers; these include, for example, polyvinylpyrrolidone, polyvinylcaprolactam, copolymers of N-vinylpyrrolidone and vinyl acetate and/or vinyl propionate in various concentration ratios, polyvinylcaprolactam, polyvinylamides and salts thereof, and copolymers of vinylpyrrolidone and dimethylaminoethyl methacrylate, terpolymers of vinylcaprolactam, vinylpyrrolidone and dimethylaminoethyl methacrylate;
polysiloxanes;
homopolymers of N-vinylformamide e.g. PVF from National Starch.

Particularly preferred nonionic polymers are acrylic acid ester copolymers, homopolymers and copolymers of vinylpyrrolidone and polyvinylcaprolactam.

Very particularly preferred nonionic polymers are homopolymers of vinylpyrrolidone, e.g. Luviskol® K from BASF, copolymers of vinylpyrrolidone and vinyl acetate, e.g. Luviskol® VA grades from BASF or PVPVA® S630L from ISP, terpolymers of vinylpyrrolidone, vinyl acetate and propionate, such as, for example, Luviskol® VAP from BASF and polyvinylcaprolactams, e.g. Luviskol® PLUS from BASF.

Advantageous anionic polymers are homopolymers or copolymers with monomer units containing acid groups which are optionally copolymerized with comonomers which contain no acid groups. Suitable monomers are unsaturated, free-radically polymerizable compounds which have at least one acid group, in particular carboxylic acid, sulphonic acid or phosphonic acid.

Advantageous anionic polymers comprising carboxylic acid groups are:
acrylic acid or methacrylic acid homopolymers or copolymers or the salts thereof. These include, for example, the copolymers of acrylic acid and acrylamides and/or sodium salts thereof, copolymers of acrylic acid and/or methacrylic acid and an unsaturated monomer selected from ethylene, styrene, vinyl ester, acrylic acid ester, methacrylic acid ester, optionally ethoxylated compounds, copolymers of vinylpyrrolidones, acrylic acid and C1-C20 alkyl methacrylates, e.g. Acrylidone® LM from ISP, copolymers of methacrylic acid, ethyl acrylates and tert-butyl acrylates, e.g. Luvimer® 100 P from BASF;
crotonic acid derivative homopolymers or copolymers or the salts thereof. These include, for example, vinyl acetate/crotonic acid, vinyl acetate/acrylate and/or vinyl acetate/vinyl neodecanoate/crotonic acid copolymers and sodium acrylate/vinyl alcohol copolymers;
unsaturated C4-C8 carboxylic acid derivatives or carboxylic anhydride copolymers selected from copolymers of maleic acid or maleic anhydride or fumaric acid or fumaric anhydride or itaconic acid or itaconic anhydride and at least one monomer selected from vinyl esters, vinyl ethers, vinylhalogen derivatives, phenylvinyl derivatives, acrylic acid, acrylic acid esters or copolymers of maleic acid or maleic anhydride or fumaric acid or fumaric anhydride or itaconic acid or itaconic anhydride and at least one monomer selected from allyl esters, methallyl esters and optionally acrylamides, methacrylamides, alpha-olefin, acrylic acid esters, methacrylic acid esters, vinylpyrrolidones. Other preferred polymers are methyl vinyl ether/maleic acid copolymers which are formed by hydrolysis of vinyl ether/maleic anhydride copolymers. These polymers may also be partially esterified (ethyl, isopropyl or butyl esters) or partially amidated.
water-soluble or -dispersible anionic polyurethanes, e.g. Luviset® PUR from BASF, which are different from the polyurethanes according to the invention,
   where this list is of course not intended to be limiting.

Advantageous anionic polymers comprising sulphonic acid group are salts of polyvinylsulphonic acid, salts of polystyrenesulphonic acid, such as, for example, sodium polystyrene sulphonate or salts of polyacrylamidosulphonic acid.

Particularly advantageous anionic polymers are acrylic acid copolymers, crotonic acid derivative copolymers, copolymers of maleic acid and maleic anhydride or fumaric acid and fumaric anhydride or itaconic acid and itaconic anhydride and at least one monomer selected from vinyl esters, vinyl ethers, vinyl halogen derivatives, phenylvinyl derivatives, acrylic acid, acrylic acid esters and salts of polystyrene sulphonic acid.

Very particularly advantageous anionic polymers are acrylate copolymers, e.g. Luvimer from BASF, ethyl acrylate/N-tert-butylacrylamide/acrylic acid copolymers ULTRAHOLD® STRONG from BASF, VA/crotonate/vinyl neodecanoate copolymer, e.g. Resyn 28-2930 from National Starch, copolymers such as, for example, copolymers of methyl vinyl ether and maleic anhydride partially esterified e.g. GANTREZ® from ISP and sodium polystyrene sulphonates, e.g. Flexan 130 from National Starch.

Advantageous amphoteric polymers can be selected from the polymers which contain units A and B distributed randomly in the polymer chain, where A is a unit which is derived from a monomer with at least one basic nitrogen atom, and B is a unit which originates from an acidic monomer which has one or more carboxy groups or sulphonic acid groups, or A and B may be groups which are derived from zwitterionic carboxybetaine monomers or sulphobetaine monomers; A and B can also be a cationic polymer chain which contains primary, secondary, tertiary or quaternary groups, in which at least one amino group carries a carboxy group or sulphonic acid group which is bonded via a hydrocarbon group, or B and C are part of a polymer chain with ethylene-α,β-dicarboxylic acid unit in which the carboxylic acid groups have been reacted with a polyamine which contains one or more primary or secondary amino groups.

Particularly advantageous amphoteric polymers are:
polymers which are formed during the copolymerization of a monomer derived from a vinyl compound with carboxy group, such as, in particular, acrylic acid, methacrylic acid, maleic acid, α-chloroacrylic acid, and a basic monomer which is derived from a vinyl compound which is substituted and comprises at least one basic atom, such as, in particular, dialkylaminoalkyl methacrylate and acrylate, dialkylaminoalkylmethacrylamide and -acrylamide. Such compounds have been described in the U.S. Pat. No. 3,836,537.
polymers with units which are derived from: a) at least one monomer which is selected from the acrylamides or methacrylamides which are substituted on the nitrogen atom with an alkyl group, b) at least one acidic comonomer which contains one or more reactive carboxy groups, and c) at least one basic comonomer, such as esters of acrylic acid and methacrylic acid with primary, secondary, tertiary and quaternary amino substituents and the quaternization product of dimethylaminoethyl methacrylate with dimethyl sulphate or diethyl sulphate.
N-substituted acrylamides or methacrylamides particularly preferred according to the invention are compounds whose alkyl groups contain 2 to 12 carbon atoms, particularly N-ethylacrylamide, N-t-butylacrylamide, N-t-octylacrylamide, N-octylacrylamide, N-decylacrylamide, N-dodecylacrylamide and the corresponding methacrylamides.
The acidic comonomers are selected in particular from acrylic acid, methacrylic acid, crotonic acid, itaconic acid, maleic acid, fumaric acid and the alkyl monoesters having 1 to 4 carbon atoms of maleic acid, maleic anhydride, fumaric acid or fumaric anhydride.
Preferred basic comonomers are aminoethyl methacrylate, butylaminoethyl methacrylate, N,N-dimethylaminoethyl methacrylate, N-t-butylaminoethyl methacrylate.
Crosslinked and completely or partially acylated polyaminoamides which are derived from polyaminoamides of the following general formula:

—[CO—R—CO—Z]—
in which R is a divalent group which is derived from a saturated dicarboxylic acid, an aliphatic mono- or dicarboxylic acid with ethylenic double bond, an ester of these acids with a lower alkanol having 1 to 6 carbon atoms or a group which is formed during the addition of one of these acids onto a bis primary or bis secondary amine, and Z is a group which is derived from a bis-primary, mono- or bis-secondary polyalkylenepolyamine, and preferably: a) in quantitative fractions of from 60 to 100 mol % the groups —NH—[(CH₂)ₓ—NH—]ₚ— where x=2 and p=2 or 3 or x=3 and p=2, where this group is derived from diethylenetriamine, triethylenetetramine or dipropylenetriamine; b) in quantitative fractions of from 0 to 40 mol % the group —NH—[(CH₂)ₓ—NH—]ₚ—, in which x=2 and p=1, which is derived from ethylenediamine, or the group which originates from piperazine:

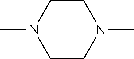

c) in quantitative fractions of from 0 to 20 mol %, the group —H—(CH₂)₆—NH—, which is derived from hexamethylenediamine, where these polyaminoamides are crosslinked by addition of a bifunctional crosslinking agent which is selected from the epihalohydrins, diepoxides, dianhydrides and bis-unsaturated derivatives, in an amount of from 0.025 to 0.35 mol of crosslinking agent per amino group of the polyaminoamide and are acylated with acrylic acid, chloroacetic acid or an alkanesulphone or salts thereof.
The saturated carboxylic acids are preferably selected from the acids having 6 to 10 carbon atoms, such as adipic acid, 2,2,4-trimethyladipic acid and 2,4,4,-trimethyladipic acid, terephthalic acid; acids with ethylenic double bond, such as, for example, acrylic acid, methacrylic acid and itaconic acid.
The alkanesultones used in the acylation are preferably propanesultone or butanesultone, the salts of the acylating agents are preferably the sodium salts or potassium salts.
Polymers with zwitterionic units of the following formula:

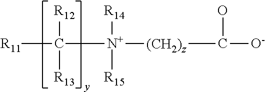

in which R₁₁ is a polymerizable unsaturated group, such as acrylate, methacrylate, acrylamide or methacrylamide, y and z are integers from 1 to 3, R₁₂ and R₁₃ are a hydrogen atom, methyl, ethyl or propyl, R₁₄ and R₁₅ are a hydrogen atom or an alkyl group which is selected such that the sum of the carbon atoms R₁₄ and R₁₅ does not exceed 10.
Polymers which contain such units can also have units which originate from non-zwitterionic monomers, such as dimethyl- and diethylaminoethyl acrylate or dimethyl- and diethylaminoethyl methacrylate or alkyl acrylates or alkyl methacrylates, acrylamides or methacrylamides or vinyl acetate.
Polymers which are derived from chitosan and contain monomer units which correspond to the following formulae:

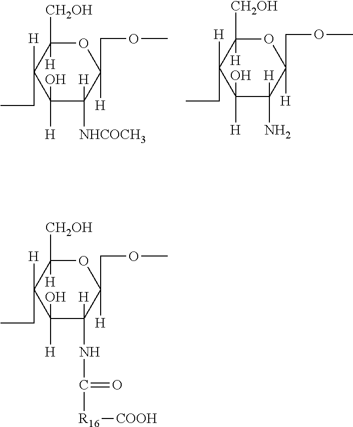

where the first unit is present in quantitative fractions of from 0 to 30%, the second unit is present in quantitative fractions of from 5 to 50% and the third unit is present in quantitative fractions of from 30 to 90%, with the proviso that in the third unit R₁₆ is a group of the following formula:

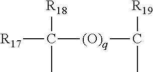

in which: if q=0, the groups R₁₇, R₁₈ and R₁₉, which are identical or different, are in each case a hydrogen atom, methyl, hydroxy, acetoxy or amino, a monoalkylamine radical or a dialkylamine radical which optionally interrupted by one or more nitrogen atoms and/or optionally by one or more groups, amino, hydroxy, carboxy, alkylthio, sulphonic acid, alkylthio, the alkyl group of which carries an amino radical, where at least one of the groups R₁₇, R₁₈ and R₁₉ is in this case a hydrogen atom; or if q=1, the groups R₁₇, R₁₈ and R₁₉ are in each case a hydrogen atom, and the salts which form these compounds with bases or acids.
Polymers which correspond to the following general formula and which are described, for example, in the French patent 1 400 366:

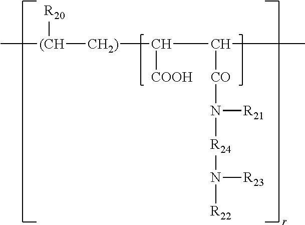

in which R₂₀ is a hydrogen atom, CH₃O, CH₃CH₂O or phenyl, R₂₁ is a hydrogen atom or a lower alkyl group, such as methyl or ethyl, R₂₂ is a hydrogen atom or a lower C₁₋₆-alkyl group, such as methyl or ethyl, R₂₃ is a lower C₁₋₆-alkyl group, such as methyl or ethyl or a group of the formula: —R₂₄—N(R₂₂)₂, where R₂₄ is a group —CH₂—CH₂, —CH₂—CH₂—CH₂— or —CH₂—CH(CH₃)— and where R₂₂ has the meanings given above.
Polymers which can be formed during the N-carboxyalkylation of chitosan, such as N-carboxymethylchitosan or N-carboxybutylchitosan.
Amphoteric polymers of the type -D-X-D-X, which are selected from: a) polymers which are formed by the action of chloroacetic acid or sodium chloroacetate on compounds with at least one unit -D-X-D-X:
in which D is the group

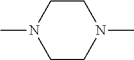

and X is the symbols E or E′, where E or E′, which are identical or different, are a divalent group which is a straight-chain or branched alkylene group having up to 7 carbon atoms in the main chain which is present in unsubstituted form or is substituted by hydroxy groups and can contain one or more oxygen atoms, nitrogen atoms or sulphur atoms and 1 to 3 aromatic and/or heterocyclic rings; where the oxygen atoms, nitrogen atoms and sulphur atoms are present in the form of the following groups: ether, thioether, sulphoxide, sulphone, sulphonium, alkylamine, alkenylamine, hydroxy, benzylamine, amine oxide, quaternary ammonium, amide, imide, alcohol, ester and/or urethane.
b) polymers of the formula -D-X-D-X, in which D is the group

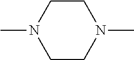

and X is the symbol E or E′ and at least once E′; where E has the meanings given above and E′ is a divalent group which is a straight-chain or branched alkylene group having up to 7 carbon atoms in the main chain, which is present in unsubstituted form or is substituted by one or more hydroxy groups and contains one or more nitrogen atoms, where the nitrogen atom is substituted by an alkyl group which is optionally interrupted by an oxygen atom and obligatorily comprises one or more carboxy functions or one or more hydroxy functions and is betainized through reaction with chloroacetic acid or sodium chloroacetate.
Alkyl(C₁₋₅) vinyl ether/maleic anhydride copolymers which in part are partially modified by semiamidation with an N,N-dialkylaminoalkylamine, such as N,N-dimethylaminopropylamine or an N,N-dialkylaminoalcohol. These polymers can also contain further comonomers, such as vinylcaprolactam.

Very particularly advantageous amphoteric polymers are, for example, the copolymers octylacrylamide/acrylates/butylaminoethyl methacrylate copolymers which are commercially available under the names AMPHOMER®, AMPHOMER® LV 71 or BALANCE® 47 from NATIONAL STARCH, and methyl methacrylate/methyl dimethylcarboxymethylammonium ethyl methacrylate copolymers.

### Solvents

The cosmetically acceptable medium of the compositions according to the invention can be water and optionally a cosmetically water-miscible suitable organic solvent.

The water used in the compositions according to the invention can be a floral water, pure demineralized water, mineral water, thermal water and/or seawater.

The preferred solvents are, for example, the aliphatic alcohols having C1-4 carbon atoms, such as ethanol and isopropanol; polyol and derivatives thereof, such as propylene glycol, dipropylene glycol, butylene-1,3 glycol, polypropylene glycol, glycol ethers such as alkyl(C1-4) ethers of mono-, di- or tripropylene glycol or mono-, di- or triethylene glycol, and mixtures thereof.

### Propellant Gases

If appropriate, the compositions according to the invention comprise propellant gases. The propellant gases preferred according to the invention are hydrocarbons such as propane, isobutene and n-butane, and mixtures thereof. However, compressed air, carbon dioxide, nitrogen, nitrogen dioxide and dimethyl ether, and mixtures of all of these gases, are also to be used advantageously according to the invention.

The person skilled in the art is naturally aware that there are propellant gases which are nontoxic per se which would in principle be suitable for realising the present invention in the form of aerosol preparations, but which nevertheless have to be dispensed with on account of a harmful effect on the environment or other accompanying phenomena, in particular fluorocarbons and chlorofluorocarbons (CFCs) such as, for example, 1,2-difluoroethane (propellant 152 A).

### Active Ingredients

The cosmetic compositions according to the invention expediently comprise one or more cosmetically effective, if appropriate also pharmaceutically effective ingredients.

Examples of cosmetically, if appropriate also therapeutically, effective ingredients include: antiacne agents, antimicrobial agents, antiperspirants, astringents, deodorizing agents, conditioners for the skin, skin-smoothing agents, agents for increasing skin hydration, such as, for example, glycerol or urea (so-called humectants), keratolytics, free-radical scavengers for free radicals, antiseptic active ingredients, active ingredients to combat skin ageing and/or agents which modulate the differentiation and/or proliferation and/or pigmentation of the skin, vitamins, such as vitamin C, active ingredients with an irritative secondary effect, such as alpha-hydroxy acids, β-hydroxy acids, alpha-keto acids, β-keto acids, retinoids (retinol, retinal, retinoic acid), anthralines (dioxyanthranol), anthranoids, peroxides (in particular benzoyl peroxide), minoxidil, lithium salts, antimetabolites, vitamin D and its derivatives; catechins, flavonoids, ceramides, fatty substances, synthetic oils, mineral oils, such as paraffin oils or Vaseline oils, silicone oils, plant oils such as coconut oil, sweet almond oil, apricot oil, corn oil, jojoba oil, olive oil, avocado oil, sesame oil, palm oil, eucalyptus oil, rosemary oil, lavender oil, pine oil, thyme oil, mint oil, cardamom oil, orange blossom oil, soybean oil, bran oil, rice oil, rapeseed oil and castor oil, wheat germ oil and vitamin E isolated therefrom, evening primrose oil, plant lecithins (e.g. soya lecithin), sphingolipids/ceramides isolated from plants, animal oils or fats, such as tallow, lanolin, butter oil, fatty acid esters, esters of fatty alcohols and waxes with a melting point corresponding to skin temperature (animal waxes, such as beeswax, carnauba wax and candelilla wax, mineral waxes, such as microcrystalline waxes, and synthetic waxes, such as polyethylene waxes or silicone waxes), and all oils suitable for cosmetic purposes, as mentioned, for example, in the CTFA publication, Cosmetic Ingredient Handbook, 1^{st} edition, 1988, The Cosmetic, Toiletry and Fragrance Association, Inc., Washington, polyunsaturated fatty acids, essential fatty acids (e.g. gamma-linolenic acid), enzymes, coenzymes, enzyme inhibitors, hydrating agents, skin-calming agents, detergents or foam-forming agents, and inorganic or synthetic matting fillers, with the exception of the nonadvantageous aforementioned non-filming fillers, abrasive agents.

Furthermore, the cosmetic compositions according to the invention can comprise plant active ingredient extracts or extracts or individual substances obtained therefrom, such as those which are selected from the group consisting of solid plant extracts, liquid plant extracts, hydrophilic plant extracts, lipophilic plant extracts, individual plant ingredients; and also mixtures thereof, such as flavonoids and their aglyca: rutin, quercetin, diosmin, hyperoside, (neo)hesperidine, hesperitine, ginkgo biloba (e.g. ginkoflavone glycosides), crataegus extract (e.g. oligomeric procyanidines), buck wheats (e.g. rutin), Sophora japonica (e.g. rutin), birch leaves (e.g. quercetin glycosides, hyperoside and rutin), elder flowers (e.g. rutin), linden blossom (e.g. essential oil with quercetin and farnesol), St. John's wort oil, (e.g. olive oil extract), calendula, arnica (e.g. oily extracts of the flowers with essential oil, polar extracts with flavonoids), Melissa (e.g. flavones, essential oil); immunostimulants: Echinacea purpurea (e.g. alcoholic extracts, fresh plant juice, pressed juice), Eleutherokokkus senticosus; alkaloids: rauwolfia (e.g. prajmalin), periwinkle (e.g. vincamin); further phytopharmaceuticals: aloe, horsechestnut (e.g. aescin), garlic (e.g. garlic oil), pineapple (e.g. bromelains), ginseng (e.g. ginsenosides), Our Lady's thistle fruit (e.g. extract standardized with regard to silymarin), box holly root (e.g. ruscogenin), valerian (e.g. valepotriates, Tct. Valerianae), cava cava (e.g. cava lactones), hop flowers (e.g. hop bitters), extr. Passiflorae, gentian (for example ethanolic extract), anthraquinone-containing drug extracts, for example aloin-containing aloe vera juice, pollen extract, algae extracts, liquorice extracts, palm extract, galphimia (e.g. original tincture), mistletoe (e.g. aqueous-ethanolic extract), phytosterols (e.g. beta-sitosterol), verbascum (e.g. aqueous-alcoholic extract), drosera (e.g. vinum liquorosum extract), sea buckthorn fruit (e.g. juice obtained therefrom or sea buckthorn oil), marshmallow root, primula root extract, fresh plant extracts of mallow, comfrey, ivy, horsetail, yarrow, ribwort (e.g. pressed juice), stinging nettle, greater celandine, parsley; plant extracts of Norolaena lobata, Tagetes lucida, Teeoma siems, Momordica charantia, and aloe vera extracts.

Preferred cosmetic active ingredients are natural and synthetic moisturizing factors and/or humectants, such as, for example, glycerol, polyglycerol, sorbitol, dimethyl isosorbide, lactic acid and/or lactates, in particular sodium lactate, butylene glycol, propylene glycol, biosaccharide gum-1, glycine soya, hydroxyethylurea, ethylhexyloxyglycerol, pyrrolidonecarboxylic acid and urea, polymeric moisturizers from the group of water-soluble and/or water-swellable and/or water-gellable polysaccharides, hyaluronic acid, chitosan, fucose-rich polysaccharides, which are obtainable under the name Fucogel™ 1000 from SOLABIA S.A., furthermore ceramides, skin protectants, skin lighteners, vitamins, antioxidants, so-called antiageing agents, anti-irritative agents etc. Further preferred cosmetic active ingredients are natural fats and oils, i.e. triglycerides of natural fatty acids, for example on account of their refatting and care effect on the skin.

Within the context of the present invention, water-soluble antioxidants can be used particularly advantageously, such as, for example, vitamins, e.g. ascorbic acid and derivatives thereof. Vitamin E and derivatives thereof, and vitamin A and derivatives thereof are very particularly advantageous.

Further advantageous active ingredients in the composition according to the invention are α-hydroxy acid such as glycolic acid, lactic acid, malic acid, tartaric acid, citric acid and mandelic acid, β-hydroxy acid such as salicylic acid, and acylated derivatives thereof, 2-hydroxyalkanoic acid and its derivatives; natural active ingredients and/or derivatives thereof, such as, for example, alpha-lipoic acid, folic acid, phytoene, D-biotin, coenzyme Q10, alpha-glucosylrutin, carnitine, carnosine, natural and/or synthetic isoflavonoids, creatine, creatinine, taurine and/or [beta]-alanine, and 8-hexadecene-1,16-dicarboxylic acid (dioic acid, CAS number 20701-68-2; provisional INCI name octadecenedioic acid) and/or licochalcone A and the plant extracts.

Pharmaceutical and therapeutic active ingredients are those which, within the context of the Drugs Law, are inter alia intended to heal, to alleviate or to prevent illnesses, suffering, bodily injury or pathological complaints. The agents and/or active ingredients are intended for external use where the active ingredients are skin-active ingredients or else transdermal active ingredients. They include, for example: compositions for the treatment of skin diseases, such as antibacterial active ingredients, antimycotics, antiviral active ingredients, anti-inflammatory active ingredients, such as dexpanthenol, itch-alleviating active ingredients, cortisone and derivatives, such as glucocorticoids, such as prednisone, prednisolone, methylprednisolone, betamethasone, dexamethasone, triamcinolone, paramethasone and fludrocortisone, agents for the treatment of skin diseases, such as neurodermatitis, atropic dermatitis etc., and antiherpes agents.

### Further Auxiliaries

The compositions according to the invention can additionally comprise supplements which are customary in cosmetics, such as antioxidants, photoprotective agents and/or other auxiliaries and supplements, such as, for example, emulsifiers, interface-active substances, antifoams, thickeners, surfactants, active ingredients, humectants, fillers, UV filters, film formers, solvents, coalescing agents, aroma substances, odour absorbers, perfumes, gelling agents and/or other polymer dispersions, such as, for example, dispersions based on polyacrylates, sensory additives, emollients, pigments, buffers, propellants, flow agents and/or thixotropic agents, suppleness agents, softeners, preservatives. The amounts of the various supplements are known to the person skilled in the art for the range to be used and are, for example, in the range from 0.0 to 25% by weight, based on the total weight of the composition.

The cosmetic compositions according to the invention can also comprise sensory additives. Sensory additives are to be understood as meaning colourless or white, mineral or synthetic, lamellar, spherical or elongated inert particles or a nonparticulate sensory additive which, for example, further improve the sensory properties of the formulations and, for example, leave behind a velvety or silky skin feel.

The sensory additives may be present in the composition according to the invention in an amount of from 0.1 to 10% by weight, based on the total weight of the composition, and preferably 0.1 to 7%.

Advantageous particulate sensory additives within the context of the present invention are talc, mica, silicon oxide, kaolin, starch and derivatives thereof (for example tapioca starch, distarch phosphate, aluminium and sodium starch octenylsuccinate and the like), fumed silica, pigments which have neither primarily UV filter effect nor colouring effect (such as, for example, boron nitride etc.), boron nitride, calcium carbonate, dicalcium phosphate, magnesium carbonate, magnesium hydrogencarbonate, hydroxyapatites, microcrystalline celluloses, powders of synthetic polymers, such as polyamides (for example the polymers available under the trade name “Nylon®”), polyethylene, poly-β-alanines, polytetrafluoroethylene (“Teflon®”), polyacrylate, polyurethane, lauroyl lysines, silicone resin (for example the polymers obtainable under the trade name “Tospearl®” from Kobo Products Inc.), hollow particles of polyvinylidene/acrylonitriles (Expancel® from Akzo Nobel) or hollow particles of silicon oxide (Silica Beads® from MAPRECOS). Advantageous nonparticulate sensory additives can be selected from the group of dimethiconoles (e.g. Dow Corning 1503 Fluid from Dow Corning Ltd.), of silicone copolymers (e.g. divinyldimethicone/dimethicone copolymer, Dow Corning HMW 2220 from Dow Corning Ltd.) or of silicone elastomers (e.g. Dimethicone Crosspolymer, Dow Corning 9040 Silicone Elastomer Blend from Dow Corning Ltd.).

The cosmetic compositions according to the invention optionally comprise a preservative. Compositions with high water contents must be reliably protected against the buildup of germs. The most important preservatives used for this purpose are urea condensates, p-hydroxybenzoates, the combination of phenoxyethanol with methyldibromoglutaronitrile and acid preservations with benzoic acid, salicylic acid and sorbic acid.

Advantageous preservatives within the context of the present invention are, for example, formaldehyde donors (such as, for example, DMDM hydantoin, which is commercially available, for example, under the trade name Glydant® (Lonza)), iodopropyl butylcarbamates (e.g. Glycacil-L®, Glycacil-S® (Lonza), Dekaben® LMB (Jan Dekker)), parabens (alkyl p-hydroxybenzoates, such as, for example, methyl, ethyl, propyl and/or butyl paraben), dehydroacetic acid (Euxyl® K 702 from Schülke & Mayr), phenoxyethanol, ethanol, benzoic acid. So-called preservation aids, such as, for example, octoxyglycerol, glycine, soya, diol etc., can also be used advantageously.

Preservatives or preservation aids customary in cosmetics, such as dibromodicyanobutane (2-bromo-2-bromomethylglutarodinitrile), phenoxyethanol, 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol, salicylic acid and salicylates, are particularly advantageous.

The preservatives are very particularly advantageously selected from the group of iodopropyl butylcarbamates, parabens (methyl, ethyl, propyl and/or butyl paraben) and/or phenoxy ethanol.

The present invention is explained by reference to examples, although these are not to be taken as limiting. Unless stated otherwise, all of the quantitative data, fractions and percentages are based on the weight and the total amount or on the total weight of the compositions.

### EXAMPLES

### Chemicals

### Bayhydur® VP LS 2336 (Bayer MaterialScience AG, Lev., DE):

Hydrophilized polyisocyanate based on hexamethylene diisocyanate, solvent-free, viscosity ca. 6800 mPa s, isocyanate content ca. 16.2%, Bayer MaterialScience AG, Leverkusen, DE.

### Impranil® DLN (Bayer MaterialScience AG, Lev., DE):

Anionically hydrophilized, noncross-branched, aliphatic polyester polyurethane-polyurea dispersion in water with a solids content of ca. 40% Bayer MaterialScience AG, Leverkusen, DE.

### Bayhydur® VP LS 2240 (Bayer MaterialScience AG, Lev., DE):

Nonionically hydrophilized, aqueous unbranched polyisocyanate dispersion comprising blocked isocyanate groups, solids content ca. 35% strength in water/MPA/xylene (56:4.5:4.5).

### Dispercoll S 5005 (Bayer MaterialScience AG, Lev., DE):

Aqueous anionic colloidally disperse solution of amorphous silicon dioxide, solids content ca. 50% strength in water, pH ca. 9, average particle size ca. 55 nm.

### Isofoam® 16 (Petrofer-Chemie, Hildesheim, DE):

### Antifoam

The other chemicals were acquired in the fine chemicals business at Sigma-Aldrich GmbH, Taufkirchen, DE.

Unless noted otherwise, all of the percentages are based on percent by weight.

Unless noted otherwise, all of the analytical measurements refer to temperatures of 23° C.

The stated viscosities were determined by means of rotary viscometry in accordance with DIN 53019 at 23° C. using a rotary viscometer from Anton Paar Germany GmbH, Ostfildern, DE.

Unless expressly mentioned otherwise, NCO contents were determined volumetrically in accordance with DIN-EN ISO 11909.

The stated particle sizes of the aqueous dispersions were determined by means of laser correlation spectroscopy (instrument: Malvern Zetasizer 1000, Malver Inst. Limited).

The particle sizes of the particles according to the invention prepared by drying aqueous dispersions was carried out by means of optical spectroscopy at 100× magnification. For the image analysis here, image processing software (SIS GmbH, Germany) was used, the analysis was carried out at 20 different object sites.

The solid body contents of the dispersions were determined by heating a weighed-out sample to 120° C. At constant weight, solid-body content was calculated by reweighing the sample. Using the same method, the water content of the powders according to the invention was determined.

The control on free NCO groups was carried out by means of IR spectroscopy (band at 2260 cm⁻¹).

### 1) Preparation of an Aqueous Nonourea Dispersion (A)

At 30° C. and with vigorous stirring, 820.20 g of Bayhydur® VP LS 2336 and then 0.32 g of Isofoam® 16 were added to a solution of 4.1 g of triethylamine in 4952 g of deionized water and the mixture was further stirred. After 3, 6 and 9 hours, in each case a further 820.20 g of Bayhydur° VP LS 2336 and in each case then 0.32 g of Isofoam® 16 were added and the mixture was then stirred for a further 4 hours at 30° C. Then, at a reduced pressure of 200 mbar and 30° C., the mixture was stirred for a further 3 hours and the resulting dispersion was drawn off.

The resulting white aqueous dispersion had the following properties:

Particle size (LCS): 93 nm
Viscosity (viscometer, 23° C.): <50 mPas

### pH (23° C.): 7.8

### 2) Comparative Example

### Drying of an Aqueous Anionically Hydrophilized, Noncross-Branched, Aliphatic Polyester Polyurethane-Polyurea Dispersion

In a stirred apparatus with distillation attachment, 500 g of the dispersion Impranil® DLN were evacuated to ca. 100 mbar at 70° C. and the water is distilled off over the distillation attachment for ca. 3 hours. This produced a gel-like, filmed mass which had to be cut out of the apparatus.

### 3) Comparative Example

### Drying of an Aqueous, Nonionically, Hydrophilized, Noncross-Branched, Aliphatic Polyester Polyurethane Dispersion

The procedure was analogous to Example 2, but the dispersion Bayhydur® VP LS 2240 was dried. A sticky film was formed, not a processable powder.

### 4) Example According to the Invention

### Direct Drying of an Aqueous Nanourea Dispersion by Distillative Removal of the Water

The procedure was analogous to Example 2, but the nanourea dispersion from Example 1 was dried. A white, lumpy powder was formed.

Solids content: 99%

By grinding the dried sample in a malter, a fine, pourable powder was obtained.

Solids content: 99%

### 5) Example According to the Invention

### Drying of an Aqueous Nanourea Dispersion by Freeze Drying

500 g of the nanourea dispersion from Example 1 were frozen in a 2 litre round-bottomed flask in a cooling bath (ca.-78° C., mixture of dry ice and isopropanol) and attached to a freeze-drying plant. Upon subsequent evacuation, the water was removed until the sample was dry.

A nonpourable agglomerized substance was formed.

Solids content: 99%

### 6) Example According to the Invention

### Drying of an Aqueous Nanourea Dispersion by Spray-Drying

2000 g of the nanourea dispersion from Example 1 were spray-dried using a B-290 spray-drying plant from Büchi (Flawli, Switzerland). The dispersion was conveyed via a nozzle with an internal diameter of 0.7 mm, the spraying gas used was nitrogen in the outer ring of the two-material nozzle. The conveying temperature was 60° C. Deposition of the particles was carried out via a cyclone and a downstream fine particle filter.

A white powder was formed.

Solids content: 99%
Average particle size: 10 μm
Bulk density: 0.36 g/ml

### 7) Example According to the Invention

### Drying of a Dilute, Aqueous Nanourea Dispersion by Spray-Drying

The procedure was as described in Example 6, but before drying a further 1000 g of deionized water were mixed into 1000 g of the nanourea dispersion.

A white powder was formed.

Solids content: 99%
Average particle size: 10 μm

### 8) Example According to the Invention

### Drying of an Aqueous Nanourea Dispersion after Mixing with a Nanosilica Dispersion by Spray-Drying

The procedure was as described in Example 6, but before the drying, 24 g of Dispercoll S 5005 were mixed into 1000 g of the nanourea dispersion.

A white powder was formed.

Solids content: 99%

### Example Formulations

### Polyurea Powders and/or Aqueous Nanourea Dispersions in Cosmetic Formulations

### 1. Skincare Formulations

### 1.1 O/W Emulsion

| | 1 | 2 | 3 | 4 | 5 | 6 | 7 | 8 | 9 | 10 |
|---|---|---|---|---|---|---|---|---|---|---|
| Polyurea powder | 5.0 | | 8.0 | 10 | 15.0 | 2.0 | 10.0 | | 5.0 | |
| according to | | | | | | | | | | |
| the invention | | | | | | | | | | |
| Aqueous nanourea | | 10 | | | | 2.0 | | 20 | | 5.0 |
| dispersion according | | | | | | | | | | |
| to the invention | | | | | | | | | | |
| Glyceryl stearate | 2.0 | | | | | | | | | |
| citrate | | | | | | | | | | |
| Glyceryl stearate | | 2.0 | | | | | | | | |
| (self-emulsifying) | | | | | | | | | | |
| Polyglyceryl-3 | | | 2.5 | 2.0 | | | | | | |
| methylglucose | | | | | | | | | | |
| distearate | | | | | | | | | | |
| Sorbitan stearate | | | | 1.0 | | | | | | |
| PEG-40 stearate | | | | | 0.5 | | | | | |
| Glyceryl stearate | | | | | 2.5 | 0.5 | 2.0 | | 2.0 | |
| PEG-100 stearate | | | | | | | 2.0 | | | |
| Sodium stearoyl | | | | | | | | 0.5 | 0.2 | |
| glutamate | | | | | | | | | | |
| Distearyldiammonium | | | | | | | | | | 1.0 |
| chloride | | | | | | | | | | |
| Stearic acid | | 1.0 | | | | | | | | |
| Behenyl alcohol | | | | | | 1.0 | | | | |
| Cetyl alcohol | | | | | | | | | | 2.5 |
| Cetearyl alcohol | | 2.0 | | | 5.0 | 10.0 | 2.0 | | | |
| Myristyl alcohol | | 2.0 | | | | | | | | |
| Stearyl alcohol | 1.0 | | 1.0 | | | | | | 3.0 | 1.0 |
| Acrylates/C10-30 alkyl | 0.1 | | | | | | 0.8 | | 0.3 | |
| acrylate crosspolymer¹ | | | | | | | | | | |
| Ammonium | | 0.5 | | | | | | | | |
| acryloyldimethyltaurate/ | | | | | | | | | | |
| VP copolymer² | | | | | | | | | | |
| Acrylic acid/VP | | | | 0.6 | | | | 0.2 | | |
| crosspolymer³ | | | | | | | | | | |
| Carbomer | | | 0.8⁴ | | 0.3⁵ | | | | 0.5⁶ | |
| Dimethylpolysiloxane | | | | | | | | | | |
| Dicaprylyl ether | 1.0 | 3.0 | | | | | | | | 1.0 |
| Myristyl myristate | 3.0 | | | | | | | 1.0 | | |
| Octyldodecanol | 1.0 | | | 4.0 | 5.0 | 3.0 | | | 4.0 | |
| Butylene glycol | | 2.0 | | | 3.0 | | | | | |
| dicaprylate/dicaprate | | | | | | | | | | |
| C12-15 Alkyl benzoate | | 3.0 | | | 3.0 | | 5.0 | 1.0 | | |
| Isohexadecane | | | 2.0 | | | | 3.0 | | | |
| Caprylic/capric | | | | 2.0 | | | | 2.0 | | |
| triglyceride | | | | | | | | | | |
| Cyclomethicone | 4.0 | | 2.0 | | | 2.0 | | | 1.0 | |
| Dimethicone | 2.0 | | 1.0 | | | 2.0 | 5.0 | | 1.0 | |
| Mineral oil | | | | | | 5.0 | | | | 2.5 |
| Hydrogenated | | | | | | 2.0 | | | | |
| polyisobutene | | | | | | | | | | |
| Phenethyl benzoate | | | | | | | | 5.0 | | |
| Isodecyl neopentanoate | | | | | | | | | 2.0 | |
| Oenothera biennis | | | | | | | | | | 2.0 |
| Shea butter | | | | | | | | | | 2.0 |
| Butylene glycol | | | | 5.0 | | | | | | |
| Glycerol | 10 | 7.5 | 5.0 | | 3.0 | | 10 | 8.0 | 5.0 | 5.0 |
| Ethanol | 3.0 | | | | | | | | 4.0 | |
| Tapioca starch⁷ | 1.0 | | 1.0 | | | | | | | |
| Distarch phosphate | | 2.0 | | | | | | | | |
| Aluminium starch⁸ | | | | | | | | 2.0 | | |
| octenylsuccinate | | | | | | | | | | |
| Sodium starch | | | | | 2.0 | | | | | |
| octenylsuccinate⁹ | | | | | | | | | | |
| Butyl | | | | | | | | 4 | | |
| methoxydibenzoylmethane | | | | | | | | | | |
| Octocrylene | | | | | | | | 5 | | |
| Phenylbenzimidazole | | | 2 | | | | | 2 | | |
| sulphonic acid | | | | | | | | | | |
| Ethylhexyl | | | 5 | | | | | | | |
| methoxycinnamate | | | | | | | | | | |
| Trisodium EDTA | | | 1 | | | | | 1 | | |
| Active ingredients | q.s. | q.s. | q.s. | q.s. | q.s. | q.s. | q.s. | q.s. | q.s. | q.s. |
| Neutralizing agents | q.s. | q.s. | q.s. | q.s. | q.s. | q.s. | q.s. | q.s. | q.s. | q.s. |
| Dyes | q.s. | q.s. | q.s. | q.s. | q.s. | q.s. | q.s. | q.s. | q.s. | q.s. |
| Perfume | q.s. | q.s. | q.s. | q.s. | q.s. | q.s. | q.s. | q.s. | q.s. | q.s. |
| Preservative | q.s. | q.s. | q.s. | q.s. | q.s. | q.s. | q.s. | q.s. | q.s. | q.s. |
| Aqua | ad | ad | ad | ad | ad | ad | ad | ad | ad | ad |
| | 100 | 100 | 100 | 100 | 100 | 100 | 100 | 100 | 100 | 100 |

¹Pemulen TR-1, Lubrizol
²Aristoflex AVC, Clariant
³UltraThix P-100, ISP
⁴Carbopol 980, Lubrizol
⁵Carbopol Ultrez 10, Lubrizol
⁶Carbopol 981, Lubrizol
⁷Tapioca Pure, National Starch
⁸Dry Flo-PC, National Starch
⁹Cleargum CO 01, Roquette

### 1.2 W/O Emulsion

| | 1 | 2 | 3 | 4 | 5 |
|---|---|---|---|---|---|
| Polyurea powder according to | 6.0 | | 8.0 | 5.0 | 10.0 |
| the invention | | | | | |
| Aqueous nanourea dispersion | | 10.0 | | 5.0 | |
| according to the invention | | | | | |
| Polygylceryl-3 diisostearate | 1.0 | | | | |
| Polyglyceryl-2 | 3.0 | 3.0 | | | |
| dipolyhydroxystearate | | | | | |
| PEG-40 sorbitan perisostearate | | 3.0 | | | |
| Triglycerol diisostearate | | | 0.5 | | |
| Diglycerol | | | 1.5 | | |
| dipolyhydroxystearate | | | | | |
| PEG-30 dipolyhydroxystearate | | | | 0.25 | |
| PEG-22 dodecyl glycol | | | | | 5.0 |
| copolymer | | | | | |
| PEG-45 dodecyl glycol polymer | | | | | 1.0 |
| Lanolin alcohol | 1.0 | | | 0.3 | 0.5 |
| Behenyl alcohol | | | 0.5 | | |
| Caprylic/capric triglyceride | 15.0 | 15.0 | | | |
| Mineral oil | 10.0 | 8.0 | 10.0 | 8.0 | 10.0 |
| Cera microcristallina | 5.0 | | 1.0 | | |
| Dicaprylyl carbonate | 1.0 | | | | |
| Isopropyl stearate | | | | | 8.0 |
| Isopropyl palmitate | 1.0 | | | | |
| Ricinus oil | 1.0 | | | | |
| Vaseline | | | 6.0 | 5.0 | |
| Octyldodecanol | | | 1.0 | 3.0 | |
| Hydrogenated cocoglyceride | | | | 2.0 | |
| Oenothera biennis | | | | | 0.5 |
| Aluminium stearate | | | 0.3 | 0.6 | 0.5 |
| Magnesium sulphate | 0.5 | 1.0 | 0.5 | 0.5 | 0.5 |
| Sodium citrate | 0.5 | 0.3 | 0.05 | | 0.2 |
| Sodium chloride | | | | 10.0 | |
| Citric acid | | | 0.1 | 0.2 | 0.2 |
| Potassium sorbate | | | 0.15 | | 0.4 |
| Glycerol | 3.0 | 8.0 | 5.0 | 3.0 | |
| Talc | | | | 0.5 | |
| Ethanol | | | 2.0 | | |
| Active ingredients | q.s. | q.s. | q.s. | q.s. | q.s. |
| Neutralizing agents | q.s. | q.s. | q.s. | q.s. | q.s. |
| Dyes | q.s. | q.s. | q.s. | q.s. | q.s. |
| Perfume | q.s. | q.s. | q.s. | q.s. | q.s. |
| Preservative | q.s. | q.s. | q.s. | q.s. | q.s. |
| Aqua | ad | ad | ad | ad | ad |
| | 100 | 100 | 100 | 100 | 100 |

### 1.3 W/Si Emulsion

| | 1 | 2 | 3 |
|---|---|---|---|
| Polyurea powder | 5.0 | 10.0 | |
| according to | | | |
| the invention | | | |
| Aqueous nanourea | | | 30.0 |
| dispersion according | | | |
| to the invention | | | |
| Cetyl dimethicone copolyol | 2.0 | | |
| Cetyl PEG/PPG-10/1 | | 3.0 | 3.0 |
| dimethicone | | | |
| Cyclomethicone | 15.0 | 25.0 | 25.0 |
| Dimethicone | 15.0 | 5.0 | 5.0 |
| Phenyltrimethicone | 1.0 | | |
| Hydrogenated polyisobutene | | 2.0 | 2.0 |
| Dimethiconol | | 1.0 | 1.0 |
| Xanthan gum¹⁰ | 0.1 | | |
| Glycerol | 5.0 | 2.0 | 2.0 |
| Magnesium sulphate | 1.0 | | |
| Sodium chloride | | 0.7 | 0.7 |
| Citric acid | | 0.3 | 0.3 |
| Sodium citrate | | 0.9 | 0.9 |
| Potassium sorbate | | 0.3 | 0.3 |
| Active ingredients | q.s. | q.s. | q.s. |
| Neutralizing agents | q.s. | q.s. | q.s. |
| Dyes | q.s. | q.s. | q.s. |
| Perfume | q.s. | q.s. | q.s. |
| Preservative | q.s. | q.s. | q.s. |
| Aqua | ad | ad | ad |
| | 100 | 100 | 100 |

¹⁰Keltrol CG-T, CP Kelco

### 1.4 Hydro Dispersion

| | 1 | 2 | 3 | 4 | 5 |
|---|---|---|---|---|---|
| Polyurea powder according to | 5.0 | 10.0 | | 5.0 | 8.0 |
| the invention | | | | | |
| Aqueous nanourea dispersion | | | 30.0 | | |
| according to the invention | | | | | |
| Cetearyl alcohol + PEG-40 | 2.5 | | | | |
| ricinus oil - sodium cetearyl | | | | | |
| sulphate | | | | | |
| Sorbitan stearate | 1.0 | | | | |
| Ceteareth-20 | | | | 0.5 | |
| Ammonium | | | | | 1.0 |
| acryloyldimethyltaurate/VP | | | | | |
| copolymer¹¹ | | | | | |
| Acrylates/C10-30 alkyl acrylate | 0.8 | 0.3 | 0.3 | 1.0 | |
| crosspolymer¹² | | | | | |
| Xanthan gum¹³ | | 0.5 | 0.5 | | |
| Octyldodecanol | 2.0 | 2.0 | 2.0 | | 2.0 |
| Caprylic/capric Triglyceride | 3.0 | 3.0 | 3.0 | | 2.0 |
| Cyclomethicone | 4.0 | | | | 2.0 |
| Isodecyl neopentanoate | | | | 3.0 | |
| Dimethicone | | | | 2.0 | |
| Dicaprylyl carbonate | | | | | 2.0 |
| Sodium starch octenyl | 1.5 | | | | |
| succinate | | | | | |
| Tapioca starch | 3.0 | | | | 1.0 |
| Alcohol | 3.0 | | | | |
| Glycerol | 5.0 | 2.0 | 2.0 | 5.0 | 2.0 |
| Ethylhexyl methoxycinnamate | | 8.0 | 8.0 | | |
| Octocrylene | | 5.0 | 5.0 | | |
| Phenylbenzimidazolesulphonic | | 2.0 | 2.0 | | |
| acid | | | | | |
| Ethanol | | | | 5.0 | |
| Sodium starch | | | | 0.5 | |
| octenylsuccinate¹⁴ | | | | | |
| Active ingredients | q.s. | q.s. | q.s. | q.s. | q.s. |
| Neutralizing agents | q.s. | q.s. | q.s. | q.s. | q.s. |
| Dyes | q.s. | q.s. | q.s. | q.s. | q.s. |
| Perfume | q.s. | q.s. | q.s. | q.s. | q.s. |
| Preservative | q.s. | q.s. | q.s. | q.s. | q.s. |
| Aqua | Ad. | Ad. | Ad. | Ad. | Ad. |
| | 100 | 100 | 100 | 100 | 100 |

¹¹Aristoflex AVC, Clariant
¹²Pemulen TR-1, Lubrizol
¹³Keltrol CG-T, CP Kelco
¹⁴Cleargum CO 01, Roquette

### 2. Suncare

### 2.1 O/W Emulsion

| | 1 | 2 | 3 | 4 | 5 | 6 | 7 | 8 | 9 | 10 | 11 | 12 | 13 |
|---|---|---|---|---|---|---|---|---|---|---|---|---|---|
| Polyurea | 6.0 | 5.0 | | 2.0 | 15.0 | 3.0 | 10.0 | 4.0 | 5.0 | 2.0 | 5.0 | | 7.50 |
| powder | | | | | | | | | | | | | |
| according to the | | | | | | | | | | | | | |
| invention | | | | | | | | | | | | | |
| Aqueous | | | 16.0 | | | | | | 5.0 | | | 20.0 | |
| nanourea | | | | | | | | | | | | | |
| dispersion | | | | | | | | | | | | | |
| according to | | | | | | | | | | | | | |
| the invention | | | | | | | | | | | | | |
| VP/eicosene | 0.5 | | | | 2.0 | | | | | 1.5 | | | |
| copolymer | | | | | | | | | | | | | |
| PVP/hexadecane | | 1.0 | | 0.5 | | 1.0 | 0.5 | 1.0 | 1.0 | | 0.5 | 1.0 | |
| copolymer | | | | | | | | | | | | | |
| Diglycol/CHDM/ | | | 3.0 | | | | | | | | | | 2.5 |
| isophthalates/ | | | | | | | | | | | | | |
| SIP | | | | | | | | | | | | | |
| copolymer | | | | | | | | | | | | | |
| Sorbitan | | | | | | | | | | | | | 2.5 |
| laurate (and) | | | | | | | | | | | | | |
| polyglyceryl- | | | | | | | | | | | | | |
| 10 laurate | | | | | | | | | | | | | |
| Glyceryl | 2.0 | | | | | | | | | | | | |
| stearate citrate | | | | | | | | | | | | | |
| Glyceryl | | 2.0 | | | | | | | | | 2.0 | | |
| stearate (self- | | | | | | | | | | | | | |
| emulsifying) | | | | | | | | | | | | | |
| Polygyceryl-2 | | | | | | | | | | | | 0.25 | |
| dipolyhydroxy- | | | | | | | | | | | | | |
| stearate | | | | | | | | | | | | | |
| Polyglyceryl-3 | | | 2.5 | 2.0 | | | | | | | | | |
| methylglucose | | | | | | | | | | | | | |
| distearate | | | | | | | | | | | | | |
| Sorbitan | | | | 1.0 | | | | | | | | | |
| stearate | | | | | | | | | | | | | |
| Glyceryl | | | | | | | | | | | | 3.5 | |
| isostearate | | | | | | | | | | | | | |
| Isoceteth-20 | | | | | | | | | | | 0.5 | 2.0 | |
| Ceteareth-12 | | | | | | | | | | | 5.0 | | |
| PEG-40 | | | | | 0.5 | | | | | | | | |
| stearate | | | | | | | | | | | | | |
| Glyceryl | | | | | 2.5 | 0.5 | 2.0 | | 2.0 | | | | |
| stearate | | | | | | | | | | | | | |
| PEG-100 | | | | | | | 2.0 | | | | | | |
| stearate | | | | | | | | | | | | | |
| Sodium | | | | | | | | 0.5 | 0.2 | | | | |
| stearoyl | | | | | | | | | | | | | |
| glutamate | | | | | | | | | | | | | |
| Distearyl- | | | | | | | | | | 1.0 | | | |
| diammonium | | | | | | | | | | | | | |
| chloride | | | | | | | | | | | | | |
| Stearic acid | | 1.0 | | | | | | | | | | | |
| Behenyl | | | | | | 1.0 | | | | | | | |
| alcohol | | | | | | | | | | | | | |
| Cetyl alcohol | | | | | | | | | | 2.5 | 1.0 | | |
| Cetearyl | | 2.0 | | | 5.0 | 10.0 | 2.0 | | | | | | |
| alcohol | | | | | | | | | | | | | |
| Myristyl | | 2.0 | | | | | | | | | | | |
| alcohol | | | | | | | | | | | | | |
| Stearyl alcohol | 1.0 | | 1.0 | | | | | | 3.0 | 1.0 | | | |
| Ethylhexyl | | | | | | | | | | | | | 7.0 |
| stearate | | | | | | | | | | | | | |
| Acrylates/ | 0.1 | | | | | | 0.8 | | 0.3 | | | | 0.2 |
| C10-30 alkyl | | | | | | | | | | | | | |
| acrylate | | | | | | | | | | | | | |
| crosspolymer¹⁵ | | | | | | | | | | | | | |
| Ammonium | | 0.5 | | | | | | | | | | | |
| acryloyldimethyltaurate/ | | | | | | | | | | | | | |
| VP | | | | | | | | | | | | | |
| copolymer¹⁶ | | | | | | | | | | | | | |
| Acrylic | | | | 0.6 | | | | 0.2 | | | | | |
| acid/VP | | | | | | | | | | | | | |
| crosspolymer¹⁷ | | | | | | | | | | | | | |
| Carbomer | | | 0.8¹⁸ | | 0.3¹⁹ | | | | 0.5²⁰ | | | | |
| Xanthan gum | | | | | | | | | | | | | 0.15 |
| Dimethylpoly- | | | | 5 | | | | | | | | | |
| siloxane | | | | | | | | | | | | | |
| Dicaprylyl | 1.0 | 3.0 | | | | | | | | 1.0 | | | |
| ether | | | | | | | | | | | | | |
| Myristyl | 3.0 | | | | | | | 1.0 | | | | | |
| myristate | | | | | | | | | | | | | |
| Octyldodecanol | 1.0 | | | 4.0 | 5.0 | 3.0 | | | 4.0 | | 3.0 | | |
| Butylene | | 2 | | 2.0 | 3.0 | | | 2 | | 7.0 | | | |
| glycol | | | | | | | | | | | | | |
| dicaprylate/ | | | | | | | | | | | | | |
| dicaprate | | | | | | | | | | | | | |
| C12-15 alkyl | | 2 | | | 3.0 | 1 | 5.0 | 2 | 5.0 | | | 5.0 | 6.0 |
| benzoate | | | | | | | | | | | | | |
| Isohexadecane | | | 2.0 | | | | 3.0 | | | | | | 1.5 |
| Caprylic/capric | | | | 2.0 | | | | 2.0 | | | | | |
| triglyceride | | | | | | | | | | | | | |
| Cyclomethicone | 4.0 | | 2.0 | | | 2.0 | | | 1.0 | | | | |
| Dimethicone | 2.0 | | 1.0 | | | 2.0 | 5.0 | | 1.0 | | | | |
| Mineral oil | | | | | | 5.0 | | | | 2.5 | | | |
| Hydrogentated | | | | | | 2.0 | | | | | | | |
| polyisobutene | | | | | | | | | | | | | |
| Phenethyl | | | | | | | | 5.0 | | | | | |
| benzoate | | | | | | | | | | | | | |
| Isodecyl | | | | | | | | | 2.0 | | | | |
| neopentanoate | | | | | | | | | | | | | |
| Oenothera | | | | | | | | | | 2.0 | | | |
| biennis | | | | | | | | | | | | | |
| Shea butter | | | | | | | | | | 2.0 | | | |
| Butylene | | | | 5.0 | | | | | | | | | |
| glycol | | | | | | | | | | | | | |
| Glycerol | 10 | 7.5 | 5.0 | | 3.0 | | 10 | 8.0 | 5.0 | 5.0 | 5.0 | 7.0 | 5.0 |
| Ethylhexyl- | 0.5 | | | | | 0.5 | | | | | | | |
| glycerol | | | | | | | | | | | | | |
| Ethanol | 3.0 | | | | | | | | 4.0 | | | | |
| Tapioca | 1.0 | | 1.0 | | | | | | | | | | |
| starch²¹ | | | | | | | | | | | | | |
| Distarch | | 2.0 | | | | | | | | | | | |
| phosphate | | | | | | | | | | | | | |
| Aluminium | | | | | | | | 2.0 | | | | | |
| starch²² | | | | | | | | | | | | | |
| octenyl- | | | | | | | | | | | | | |
| succinate | | | | | | | | | | | | | |
| Sodium starch | | | | | 2.0 | | | | | | | | |
| octenyl- | | | | | | | | | | | | | |
| succinate²³ | | | | | | | | | | | | | |
| Bisethyl- | | | 1 | | | | | | 2.0 | 3.0 | | | |
| hexyloxy | | | | | | | | | | | | | |
| phenol | | | | | | | | | | | | | |
| methoxyphenyl | | | | | | | | | | | | | |
| triazine | | | | | | | | | | | | | |
| Ethylhexyl | | | | 2.0 | | | | | 2.0 | | | | |
| triazone | | | | | | | | | | | | | |
| Butyl | 3 | 3 | | | 2.5 | 4 | | 2 | 3.0 | 1.0 | 2.0 | 3.0 | 2.0 |
| methoxy- | | | | | | | | | | | | | |
| dibenzoyl- | | | | | | | | | | | | | |
| methane | | | | | | | | | | | | | |
| Ethylhexyl | 4 | 4 | 3 | | 5.0 | | 5.0 | 5 | 5.0 | | 5.0 | | 7.0 |
| methoxy- | | | | | | | | | | | | | |
| cinnamate | | | | | | | | | | | | | |
| Octocrylene | | | | | 4.0 | 5 | | | | | | 4.0 | |
| Phenylbenzimidazole | | | | 1.0 | 2.0 | 2 | 2.0 | | | | | | 4.0 |
| sulphonic acid | | | | | | | | | | | | | |
| Titanium | | | | | | 1 | | | | | | | |
| dioxide + | | | | | | | | | | | | | |
| trimethoxy- | | | | | | | | | | | | | |
| caprylylsilane | | | | | | | | | | | | | |
| Terephthalidene | | | | | | | | | | | | 3.0 | |
| dicamphor | | | | | | | | | | | | | |
| sulphonic acid | | | | | | | | | | | | | |
| Diethylamino | | | | | | | 1.0 | | | | | | |
| hydrobenzoyl | | | | | | | | | | | | | |
| hexylbenzoate | | | | | | | | | | | | | |
| Phenylene-1,4- | | | | 1.0 | | | 2.0 | | | | | | |
| bis-(2- | | | | | | | | | | | | | |
| benzimidazole)- | | | | | | | | | | | | | |
| 3,3,5,5′- | | | | | | | | | | | | | |
| tetrasulphonic | | | | | | | | | | | | | |
| acid | | | | | | | | | | | | | |
| Polysilicone- | | | | | | | | | | 3.0 | | | |
| 15 | | | | | | | | | | | | | |
| Benzophenone-3 | | | | | | | | | | | 2.0 | | |
| Titanium | | | | 2.0 | | | | | | | | | |
| dioxide | | | | | | | | | | | | | |
| Trisodium | 1 | 1 | 1 | | | | 1 | 1 | 1 | 1 | 1 | | 1 |
| EDTA | | | | | | | | | | | | | |
| Active | q.s. | q.s. | q.s. | q.s. | q.s. | q.s. | q.s. | q.s. | q.s. | q.s. | q.s. | q.s. | q.s. |
| ingredients | | | | | | | | | | | | | |
| Neutralizing | q.s. | q.s. | q.s. | q.s. | q.s. | q.s. | q.s. | q.s. | q.s. | q.s. | q.s. | q.s. | q.s. |
| agents | | | | | | | | | | | | | |
| Dyes | q.s. | q.s. | q.s. | q.s. | q.s. | q.s. | q.s. | q.s. | q.s. | q.s. | q.s. | q.s. | q.s. |
| Perfume | q.s. | q.s. | q.s. | q.s. | q.s. | q.s. | q.s. | q.s. | q.s. | q.s. | q.s. | q.s. | q.s. |
| Preservative | q.s. | q.s. | q.s. | q.s. | q.s. | q.s. | q.s. | q.s. | q.s. | q.s. | q.s. | q.s. | q.s. |
| Aqua | ad | ad | ad | ad | ad | ad | ad | ad | ad | ad | ad | ad | ad |
| | 100 | 100 | 100 | 100 | 100 | 100 | 100 | 100 | 100 | 100 | 100 | 100 | 100 |

¹⁵Pemulen TR-1, Lubrizol
¹⁶Aristoflex AVC, Clariant
¹⁷UltraThix P-100, ISP
¹⁸Carbopol 980, Lubrizol
¹⁹Carbopol Ultrez 10, Lubrizol
²⁰Carbopol 981, Lubrizol
²¹Tapioca Pure, National Starch
²²Dry Flo-PC, National Starch
²³Cleargum CO 01, Roquette

### 2.2 W/O Emulsion

| | 1 | 2 | 3 | 4 | 5 | 6 | 7 |
|---|---|---|---|---|---|---|---|
| Polyurea powder according to the | 2.0 | 5.0 | 8.0 | | | 2.0 | 10.0 |
| invention | | | | | | | |
| Aqueous nanourea dispersion | | | | 5.0 | 30.0 | | |
| according to the invention | | | | | | | |
| VP/eicosene copolymer | 1.0 | | | 1.0 | | 0.5 | |
| PVP/hexadecane copolymer | | 0.5 | 1.0 | | 1.0 | | 2.0 |
| Polygylceryl-3 diisostearate | 1.0 | | | 1.0 | | | |
| Polyglyceryl-2 | 3.0 | 3.0 | | 3.0 | | | |
| dipolyhydroxystearate | | | | | | | |
| PEG-40 sorbitan perisostearate | | 3.0 | | | | | |
| Triglycerol diisostearate | | | 0.5 | | 0.5 | | |
| Diglycerol dipolyhydroxystearate | | | 1.5 | | 1.5 | | |
| PEG-30 Dipolyhydroxystearate | | | | | | 0.25 | |
| PEG-22 dodecyl glycol copolymer | | | | | | | 5.0 |
| PEG-45 dodecyl glycol polymer | | | | | | | 1.0 |
| Lanolin alcohol | 1.0 | | | 1.0 | | 0.3 | 0.5 |
| Behenyl alcohol | | | 0.5 | | 0.5 | | |
| Caprylic/capric triglyceride | 15.0 | 15.0 | | 15.0 | | | |
| Mineral oil | 10.0 | 8.0 | 10.0 | 10.0 | 10.0 | 8.0 | 10.0 |
| Cera microcristallina | 5.0 | | 1.0 | 5.0 | 1.0 | | |
| Dicaprylyl carbonate | 1.0 | | | 1.0 | | | |
| Isopropyl stearate | | | | | | | 8.0 |
| Isopropyl palmitate | 1.0 | | | 1.0 | | | |
| Ricinus oil | 1.0 | | | 1.0 | | | |
| Vaseline | | | 6.0 | | 6.0 | 5.0 | |
| Octyldodecanol | | | 1.0 | | 1.0 | 3.0 | |
| Hydrogenated cocoglycerides | | | | | | 2.0 | |
| Oenothera biennis | | | | | | | 0.5 |
| Butylene glycol | | | 2 | | 2 | | |
| dicaprylate/dicaprate | | | | | | | |
| C12-15 alkyl benzoate | | 4 | 2 | | 2 | 1 | 4 |
| Aluminium stearate | | | 0.3 | | 0.3 | 0.6 | 0.5 |
| Magnesium sulphate | 0.5 | 1.0 | 0.5 | 0.5 | 0.5 | 0.5 | 0.5 |
| Sodium citrate | 0.5 | 0.3 | 0.05 | 0.5 | 0.05 | | 0.2 |
| Sodium chloride | | | | | | 10.0 | |
| Citric acid | | | 0.1 | | 0.1 | 0.2 | 0.2 |
| Potassium sorbate | | | 0.15 | | 0.15 | | 0.4 |
| Ethylhexylglycerol | 0.5 | | | 0.5 | | | |
| Glycerol | 3.0 | 8.0 | 5.0 | 3.0 | 5.0 | 3.0 | |
| Trisodium EDTA | | 1 | 1 | | 1 | | |
| Ethylhexyl triazone | | | | | | | |
| Butyl methoxydibenzoylmethane | | 2.5 | | | | 4 | 2 |
| Ethylhexyl methoxycinnamate | | | 3 | | 3 | | 5 |
| Octocrylene | 3 | | | 3 | | 5 | |
| Phenylbenzimidazole sulphonic acid | | 4 | | | | 2 | |
| Titanium dioxide | 1 | | | 1 | | 1 | |
| Talc | | | | | | 0.5 | |
| Ethanol | | | 2.0 | | 2.0 | | |
| Trisodium EDTA | | 1 | 1 | | 1 | | |
| Active ingredients | q.s. | q.s. | q.s. | q.s. | q.s. | q.s. | q.s. |
| Neutralizing agents | q.s. | q.s. | q.s. | q.s. | q.s. | q.s. | q.s. |
| Dyes | q.s. | q.s. | q.s. | q.s. | q.s. | q.s. | q.s. |
| Perfume | q.s. | q.s. | q.s. | q.s. | q.s. | q.s. | q.s. |
| Preservative | q.s. | q.s. | q.s. | q.s. | q.s. | q.s. | q.s. |
| Aqua | ad | ad | ad | ad | ad | ad | ad |
| | 100 | 100 | 100 | 100 | 100 | 100 | 100 |

### 2.3 W/Si Emulsion

| | 1 | 2 | 3 |
|---|---|---|---|
| Polyurea powder | 5.0 | | 10.0 |
| according to | | | |
| the invention | | | |
| Aqueous nanourea | | 10.0 | |
| dispersion according | | | |
| to the invention | | | |
| VP/eicosene copolymer | 1.0 | 1.0 | |
| PVP/hexadecane copolymer | | | 1.0 |
| Cetyl dimethicone copolyol | 2.0 | 2.0 | |
| Cetyl PEG/PPG-10/1 | | | 3.0 |
| dimethicone | | | |
| Cyclomethicone | 15.0 | 15.0 | 25.0 |
| Dimethicone | 15.0 | 15.0 | 5.0 |
| Phenyltrimethicone | 1.0 | 1.0 | |
| Hydrogenated polyisobutene | | | 2.0 |
| Dimethiconol | | | 1.0 |
| Xanthan gum²⁴ | 0.1 | 0.1 | |
| Ethylhexylglycerol | | | 0.5 |
| Glycerol | 5.0 | 5.0 | 2.0 |
| Magnesium sulphate | 1.0 | 1.0 | |
| Sodium chloride | | | 0.7 |
| Citric acid | | | 0.3 |
| Sodium citrate | | | 0.9 |
| Potassium sorbate | | | 0.3 |
| Trisodium EDTA | | | 1 |
| Ethylhexyl triazone | 2 | 2 | |
| Butyl | 3 | 3 | |
| methoxydibenzoylmethane | | | |
| Ethylhexyl methoxycinnamate | | | 2 |
| Titanium dioxide | 0.5 | 0.5 | 2 |
| Active ingredients | q.s. | q.s. | q.s. |
| Neutralizing agents | q.s. | q.s. | q.s. |
| Dyes | q.s. | q.s. | q.s. |
| Perfume | q.s. | q.s. | q.s. |
| Preservatives | q.s. | q.s. | q.s. |
| Aqua | ad | ad | ad |
| | 100 | 100 | 100 |

²⁴Keltrol CG-T, CP Kelco

### 2.4 Hydrodispersion

| | 1 | 2 | 3 | 4 | 5 | 6 |
|---|---|---|---|---|---|---|
| Polyurea powder according to | 2.5 | | 10.0 | 5.0 | 5.0 | 8.0 |
| the invention | | | | | | |
| Aqueous nanourea dispersion | | 10.0 | | | 20.0 | |
| according to the invention | | | | | | |
| VP/eicosene copolymer | 1.0 | 1.0 | | | | |
| PVP/hexadecane copolymer | | | 0.5 | | | 1.0 |
| Diglycol/CHDM/ | | | | 2.0 | 2.0 | |
| isophthalates/SIP | | | | | | |
| copolymer | | | | | | |
| Cetearyl alcohol + PEG-40 | 2.5 | 2.5 | | | | |
| ricinus | | | | | | |
| oil - sodium cetearyl sulphate | | | | | | |
| Sorbitan stearate | 1.0 | 1.0 | | | | |
| Ceteareth-20 | | | | 0.5 | 0.5 | |
| Ammonium | | | | | | 1.0 |
| acryloyldimethyltaurate/VP | | | | | | |
| Copolymer²⁵ | | | | | | |
| Acrylates/C10-30 alkyl | 0.8 | 0.8 | 0.3 | 1.0 | 1.0 | |
| acrylate | | | | | | |
| crosspolymer²⁶ | | | | | | |
| Xanthan gum²⁷ | | | 0.5 | | | |
| Octyldodecanol | 2.0 | 2.0 | 2.0 | | | 2.0 |
| Caprylic/capric triglyceride | 3.0 | 3.0 | 3.0 | | | 2.0 |
| Cyclomethicone | 4.0 | 4.0 | | | | 2.0 |
| Isodecyl neopentanoate | | | | 3.0 | 3.0 | |
| Dimethicone | | | | 2.0 | 2.0 | |
| Dicaprylyl carbonate | | | | | | 2.0 |
| Butylene glycol | 2.0 | 2.0 | | 5.0 | 5.0 | |
| dicaprylate/dicaprate | | | | | | |
| C12-15 alkyl benzoate | 2.0 | 2.0 | | | | 5.0 |
| Sodium starch | 1.5 | 1.5 | | | | |
| octenylsuccinate | | | | | | |
| Tapioca starch | 3.0 | 3.0 | | | | 1.0 |
| Ethanol | 3.0 | 3.0 | | | | |
| Glycerol | 5.0 | 5.0 | 2.0 | 5.0 | 5.0 | 2.0 |
| Ethylhexyl methoxycinnamate | 3.0 | 3.0 | 8.0 | | | |
| Octocrylene | | | 5.0 | | | |
| Phenylbenzimidazole | | | 2.0 | | | |
| sulphonic acid | | | | | | |
| Butyl | 2.0 | 2.0 | | 3.0 | 3.0 | 3.0 |
| methoxydibenzoylmethane | | | | | | |
| Ethylhexyl triazone | | | | 2.0 | 2.0 | 2.0 |
| Bis-Ethylhexyloxyphenol | | | | | | 2.0 |
| methoxyphenyl triazine | | | | | | |
| Titanium dioxide | | | | 0.5 | 0.5 | |
| Trisodium EDTA | 1.0 | 1.0 | | 1.0 | 1.0 | 1.0 |
| Ethanol | | | | 5.0 | 5.0 | |
| Sodium starch | | | | 0.5 | 0.5 | |
| octenylsuccinate²⁸ | | | | | | |
| Active ingredients | q.s. | q.s. | q.s. | q.s. | q.s. | q.s. |
| Neutralizing agents | q.s. | q.s. | q.s. | q.s. | q.s. | q.s. |
| Dyes | q.s. | q.s. | q.s. | q.s. | q.s. | q.s. |
| Perfume | q.s. | q.s. | q.s. | q.s. | q.s. | q.s. |
| Preservative | q.s. | q.s. | q.s. | q.s. | q.s. | q.s. |
| Aqua | ad | ad | ad | ad | ad | ad |
| | 100 | 100 | 100 | 100 | 100 | 100 |

²⁵Aristoflex AVC, Clariant
²⁶Pemulen TR-1, Lubrizol
²⁷Keltrol CG-T, CP Kelco
²⁸Cleargum CO 01, Roquette

### 3. Decorative Cosmetics

### 3.1 Mascara

| | % by wt. | % by wt. | % by wt. |
|---|---|---|---|
| | (based on | (based on | (based on |
| | the cosmetic | the cosmetic | the cosmetic |
| Raw materials | composition) | composition) | composition) |
| Isododecane | 20.00 | 20.00 | 20.00 |
| D5 Cyclomethicone | 5.00 | 5.00 | 5.00 |
| Carnauba wax | 6.00 | 6.00 | 6.00 |
| Trimethyl siloxysilicate | 0.75 | 0.75 | 0.75 |
| Dimethicone 200/200 | 10.00 | 10.00 | 10.00 |
| PVP/eicosene copolymer | 7.5 | 7.5 | 7.5 |
| Polyurea powder | 5.0 | 5.0 | 0 |
| according to | | | |
| the invention | | | |
| Aqueous nanourea | 0 | 5.0 | 10.0 |
| dispersion according | | | |
| to the invention | | | |
| Ceresin wax SP252 | 3.00 | 3.00 | 3.00 |
| Paraffin wax 130/135 | 3.50 | 3.50 | 3.50 |
| Polyethylene | 2.50 | 2.50 | 2.50 |
| Nylon-12 | 2.00 | 2.00 | 2.00 |
| Silica | 2.00 | 2.00 | 2.00 |
| Stearic acid | 1.00 | 1.00 | 1.00 |
| Bentone gel in | 15.00 | 15.00 | 15.00 |
| isododecane | | | |
| Phenoxyethanol | 1.00 | 1.00 | 1.00 |
| Black iron oxide | 10.00 | 10.00 | 10.00 |
| LC989 EM | | | |
| White beeswax | 1.75 | 1.75 | 1.75 |
| Deionized water | ad 100 | ad 100 | ad 100 |
| Magnesium aluminium | 0.50 | 0.50 | 0.50 |
| silicate | | | |
| Triethanolamine 99% | 0.90 | 0.90 | 0.90 |
| Net-DTB (10% in | 1.00 | 1.00 | 1.00 |
| butylene glycol) | | | |

### 3.2 Foundation

| | % by wt. | % by wt. | % by wt. |
|---|---|---|---|
| | (based on | (based on | (based on |
| | the cosmetic | the cosmetic | the cosmetic |
| Raw materials | composition) | composition) | composition) |
| Deionized water | ad. 100 | ad. 100 | ad. 100 |
| Cellulose gum | 0.30 | 0.30 | 0.30 |
| Magnesium | 0.35 | 0.35 | 0.35 |
| aluminum | | | |
| silicate | | | |
| Lecithin | 0.40 | 0.40 | 0.40 |
| Triethanolamine | 1.25 | 1.25 | 1.25 |
| 99% | | | |
| Butylene glycol | 6.00 | 6.00 | 6.00 |
| Titanium dioxide | 8.00 | 8.00 | 8.00 |
| (water | | | |
| dispersible) | | | |
| Red iron oxide | 0.40 | 0.40 | 0.40 |
| Yellow iron | 0.80 | 0.80 | 0.80 |
| oxide | | | |
| Black iron oxide | 0.10 | 0.10 | 0.10 |
| Colloidal kaolin | 2.00 | 2.00 | 2.00 |
| Methyl paraben | 0.20 | 0.20 | 0.20 |
| Isoeicosane | 10.00 | 10.00 | 10.00 |
| Isostearic acid | 1.00 | 1.00 | 1.00 |
| Stearic acid | 2.50 | 2.50 | 2.50 |
| Glyceryl stearate | 1.50 | 1.50 | 1.50 |
| Tridecyl | 1.00 | 1.00 | 1.00 |
| trimellitate | | | |
| Glyceryl stearate | 1.00 | 1.00 | 1.00 |
| SE | 0.20 | 0.20 | 0.20 |
| Propyl paraben | | | |
| Acrylates | 5.0 | 5.0 | 5.0 |
| copolymer²⁹ | | | |
| Polyurea powder | 5.0 | 2.5 | 0 |
| according to | | | |
| the invention | | | |
| Aqueous | 0 | 2.5 | 20.0 |
| nanourea | | | |
| dispersion | | | |
| Active | q.s. | q.s. | q.s. |
| ingredients | | | |
| Dyes | q.s. | q.s. | q.s. |
| Perfume | q.s. | q.s. | q.s. |
| Preservative | q.s. | q.s. | q.s. |
| Aqua | ad100 | ad100 | ad100 |

²⁹Covacryl E 14, Sensient

### 3.3 Eyeliner

| | % by wt. | % by wt. | % by wt. |
|---|---|---|---|
| | (based on | (based on | (based on |
| | the cosmetic | the cosmetic | the cosmetic |
| Raw materials | composition) | composition) | composition) |
| Oleyl alcohol | 0.5 | 0.5 | 0.5 |
| Propylene glycol | 7.5 | 7.5 | 7.5 |
| Xanthan gum | 0.1 | 0.1 | 0.1 |
| Silica | 0.1 | 0.1 | 0.1 |
| Acrylates | 0.5 | 0.5 | 0.5 |
| copolymer³⁰ | | | |
| Polyurea powder | 2.0 | 1.0 | 0 |
| according to | | | |
| the invention | | | |
| Aqueous nanourea | 0 | 1.0 | 2.0 |
| dispersion | | | |
| Active ingredients | q.s. | q.s. | q.s. |
| Dyes | q.s. | q.s. | q.s. |
| Perfume | q.s. | q.s. | q.s. |
| Preservative | q.s. | q.s. | q.s. |
| Aqua | Ad. 100 | Ad. 100 | ad. 100 |

³⁰Covacryl E 14, Sensient

### 3.4 Tanning Composition

| | % by wt. | % by wt. |
|---|---|---|
| | (based on | (based on |
| | the cosmetic | the cosmetic |
| Raw materials | composition) | composition) |
| Dihydroxyacetone | 3.0 | 3.0 |
| Glycerol | 8.0 | 8.0 |
| Cetyl alcohol | 0.5 | 0.5 |
| Silica | 3.0 | 3.0 |
| Methylglucose sesquistearate | 2.0 | 2.0 |
| PEG-100 stearate | 1.0 | 1.0 |
| Cyclomethicone | 4.0 | 4.0 |
| Polyurea powder | 10.0 | |
| according to | | |
| the invention | | |
| Aqueous nanourea | | 20.0 |
| dispersion according | | |
| to the invention | | |
| Octyldodecanol | 3.0 | 3.0 |
| Dicaprylyl carbonate | 2.0 | 2.0 |
| EDTA | 1.0 | 1.0 |
| Xanthan gum | 0.3 | 0.3 |
| Sodium citrate | 0.4 | 0.4 |
| Citric acid | 0.3 | 0.3 |
| Vitamin E acetate | 0.5 | 0.5 |
| Active ingredients | q.s. | q.s. |
| Dyes | q.s. | q.s. |
| Perfume | q.s. | q.s. |
| Preservative | q.s. | q.s. |
| Aqua | ad100 | ad100 |

### 3.5 Tinted Day Cream

| | % by wt. | % by wt. |
|---|---|---|
| | (based on | (based on |
| | the cosmetic | the cosmetic |
| Raw materials | composition) | composition) |
| Glyceryl stearate citrate | 3.5 | 3.5 |
| Octyldodecanol | 3.0 | 3.0 |
| Cyclomethicone | 3.0 | 3.0 |
| Cetearyl alcohol | 1.5 | 1.5 |
| Squalane | 2.0 | 2.0 |
| Shea butter | 5.0 | 5.0 |
| Carbomer | 0.5 | 0.5 |
| Glycerol | 10.0 | 10.0 |
| 4-Methylbenzylidene camphor | 5.0 | 5.0 |
| Octyl methoxycinnamate | 2.5 | 2.5 |
| Octocrylene | 6.0 | 6.0 |
| Butyl methoxydibenzoylmethane | 2.5 | 2.5 |
| Polyurea powder | 5.0 | |
| according to | | |
| the invention | | |
| Aqueous nanourea | | 20.0 |
| dispersion according | | |
| to the invention | | |
| EDTA | 1.0 | 1.0 |
| Active ingredients | q.s. | q.s. |
| Dyes | q.s. | q.s. |
| Perfume | q.s. | q.s. |
| Preservative | q.s. | q.s. |
| Aqua | ad 100 | ad 100 |

### 3.6 Lipstick

| | % by wt. | % by wt. | % by wt. |
|---|---|---|---|
| | (based on | (based on | (based on |
| | the cosmetic | the cosmetic | the cosmetic |
| Raw materials | composition) | composition) | composition) |
| Ricinus oil | 3.0 | 3.0 | 3.0 |
| Caprylic/capric triglycerides | 3.0 | 3.0 | 3.0 |
| Octyldodecanol | 5.0 | 5.0 | 5.0 |
| Hydrogenated polyisobutene | 3.0 | 3.0 | 3.0 |
| Jojaba oil | 1.0 | 1.0 | 1.0 |
| Lanolin oil | 1.0 | 1.0 | 1.0 |
| PEG 45/dodecyl glycol | 2.0 | 2.0 | 2.0 |
| copolymer | | | |
| Polyglyceryl-3 diisostearate | 2.4 | 2.4 | 2.4 |
| Cetyl palmitate | 1.0 | 1.0 | 1.0 |
| C20-40 alkyl stearate | 8.0 | 8.0 | 8.0 |
| Carnauba wax | 2.0 | 2.0 | 2.0 |
| Microcrystalline wax | 8.0 | 8.0 | 8.0 |
| Glycerol | 10.0 | 10.0 | 10.0 |
| Polyurea powder | 15.0 | 8.0 | |
| according to the | | | |
| invention | | | |
| Aqueous nanourea | | 8.0 | 30.0 |
| dispersion according | | | |
| to the invention | | | |
| Active ingredients | q.s. | q.s. | q.s. |
| Dyes | q.s. | q.s. | q.s. |
| Perfume | q.s. | q.s. | q.s. |
| Preservative | q.s. | q.s. | q.s. |
| Aqua | ad 100 | ad 100 | ad 100 |

| | % by wt. |
|---|---|
| | (based on |
| | the cosmetic |
| Raw materials | composition) |
| Microcrystalline wax | 3.0 |
| Candelilla wax | 12.0 |
| Cera alba | 2.5 |
| Polyglyceryl-3 diisostearate | 10.0 |
| Cyclomethicone | 25.0 |
| Octyldodecanol | Ad 100 |
| Polyisobutene | 5.0 |
| Caprylic/capric triglecerides | 12.0 |
| Pentaerythrityl tetraisostearate | 10.0 |
| PVP/hexadecene copolymer | 0.5 |
| Polyurea powder | 5.0 |
| according to | |
| the invention | |
| Active ingredients | q.s. |
| Dyes | q.s. |
| Perfume | q.s. |
| Preservative | q.s. |

### 3.7 Face Powder (Loose)

| | % by wt. |
|---|---|
| | (based on |
| | the cosmetic |
| Raw materials | composition) |
| Talc | 59.000 |
| Mica | 10.000 |
| Zinc stearate | 6.000 |
| Lauroyl lysine | 0.450 |
| Bismuth oxychloride | 5.000 |
| Nylon-12 | 5.000 |
| Silica | 5.000 |
| Yellow iron oxide | 0.750 |
| Red iron oxide | 0.375 |
| Black iron oxide | 0.075 |
| Potassium sorbate | 0.200 |
| BHT | 0.050 |
| Polyurea powder | 5.000 |
| according to | |
| the invention | |
| Ethylhexyl palmitate | 1.000 |
| Phenoxyethanol | 1.000 |
| Dimethicone | 1.000 |
| Tocopheryl acetate | 0.100 |

### 3.8 Face Powder (Pressed)

| | % by wt. |
|---|---|
| | (based on |
| | the cosmetic |
| Raw materials | composition) |
| Titanium dioxide | 5.0 |
| Magnesium stearate | 3.0 |
| Magnesium silica | 33.0 |
| Mica | 7.0 |
| Iron oxides | 2.0 |
| Talc | ad 100 |
| Isopropyl isostearate | 2.0 |
| Octyldodecyl stearoyl | 2.0 |
| stearate | |
| Polyurea powder | 10.0 |
| according to | |
| the invention | |
| Active ingredients | q.s |
| Dyes | q.s |
| Perfume | q.s |
| Preservative | q.s |

### 3.9 Cast Blusher

| | % by wt. |
|---|---|
| | (based on |
| | the cosmetic |
| Raw materials | composition) |
| Caprylic/capric triglyceride | ad 100 |
| Microcrystalline wax | 3.5 |
| Cera alba | 4.0 |
| C20-40 alkyl stearate | 6.0 |
| Cetyl palmitate | 2.5 |
| Isononyl isononanoate | 6.0 |
| Lauroyl lysin | 1.0 |
| Nylon | 4.0 |
| PVP/eicosene copolymer | 2.0 |
| Tocopheryl acetate | 1.0 |
| Polyurea powder | 10.0 |
| according to | |
| the invention | |
| Active ingredients | q.s |
| Dyes | q.s |
| Perfume | q.s |
| Preservative | q.s |

### 4. Deodorants and Antiperspirants

### 4.1 Deodorant Spray

| | % by wt. | % by wt. |
|---|---|---|
| | (based on | (based on |
| | the cosmetic | the cosmetic |
| Raw materials | composition) | composition) |
| Farnesol | 1.0 | 1.0 |
| Octyldodecanol | 0.5 | 0.5 |
| Ethanol | ad 100 | ad 100 |
| Propellant gas | 60.0 | 60.0 |
| Aqueous nanourea | | 10.0 |
| dispersion according | | |
| to the invention | | |
| Polyurea powder | 5.0 | |
| according to | | |
| the invention | | |

### 4.2 Deodorant Pumpspray

| | % by wt. |
|---|---|
| | (based on |
| | the cosmetic |
| Raw materials | composition) |
| Deodorant perfume oil | 2.5 |
| Solubility promoter | 0.5 |
| Ethanol | 60.0 |
| Water | 30 |
| PEG-100 | 2.0 |
| Polyurea powder | 5.0 |
| according to | |
| the invention | |

### 4.3 Antiperspirant Roller Gel

| | % by wt. | % by wt. | % by wt. |
|---|---|---|---|
| | (based on | (based on | (based on |
| | the cosmetic | the cosmetic | the cosmetic |
| Raw materials | composition) | composition) | composition) |
| ACH (50% by wt. aq. | 20.0 | 20.0 | 20.0 |
| solution) | | | |
| Hydroxyethylcellulose | 0.5 | 0.5 | 0.5 |
| Ethanol | 40 | 40 | 40 |
| Water | ad 100 | ad 100 | ad 100 |
| Dyes | q.s | q.s | q.s |
| Perfume | q.s | q.s | q.s |
| Solubility promoter | q.s | q.s | q.s |
| Aqueous nanourea | | 7.5 | 20.0 |
| dispersion according | | | |
| to the invention | | | |
| Polyurea powder | 7.5 | 7.5 | |
| according to | | | |
| the invention | | | |

### 4.4 Antiperspirant Stick

| | % by wt. |
|---|---|
| | (based on |
| | the cosmetic |
| Raw materials | composition) |
| Al—Zr-tetrachlorohydrex | 40.0 |
| glycerol (35% by wt. aq. | |
| solution) | |
| Stearyl alcohol | 20.0 |
| Glycerylstearate + PEG-100 | 1.0 |
| stearate | |
| Talc | 1.5 |
| PEG-20 | 5.0 |
| Aerosil | 1.5 |
| Cyclopentasiloxane | ad 100 |
| Dyes | q.s |
| Perfume | q.s |
| Solubility promoter | q.s |
| Polyurea powder | 7.5 |
| according to | |
| the invention | |

### 4.5 Deodorant Cream

| | % by wt. | % by wt. |
|---|---|---|
| | (based on | (based on |
| | the cosmetic | the cosmetic |
| Raw materials | composition) | composition) |
| Glyceryl stearate | 4.0 | 4.0 |
| PEG-2000 stearate | 4.5 | 4.5 |
| Cetyl alcohol | 5.0 | 5.0 |
| Cyclopentasiloxane | 6.0 | 6.0 |
| Mineral oil | 4.5 | 4.5 |
| Avocado oil | 0.1 | 0.1 |
| Water | ad 100 | ad 100 |
| Preservative | q.s. | q.s. |
| Dyes | q.s | q.s |
| Deodorant perfume oil | q.s | q.s |
| Solubility promoter | q.s | q.s |
| Aqueous nanourea | | 15 |
| dispersion according | | |
| to the invention | | |
| Polyurea powder | 10 | |
| according to | | |
| the invention | | |

### 4.6 Roller Deodorant O/W Emulsion

| | % by wt. | % by wt. |
|---|---|---|
| | (based on | (based on |
| | the cosmetic | the cosmetic |
| Raw materials | composition) | composition) |
| Glyceryl laurate | 1.0 | 1.0 |
| Glyceryl stearate | 4.0 | 4.0 |
| Cetyl alcohol | 3.0 | 3.0 |
| Octyldodecanol | 5.0 | 5.0 |
| Ethanol | 10.0 | 10.0 |
| Glycerol | 5.0 | 5.0 |
| Carbomer³¹ | 0.6 | 0.6 |
| Water | ad 100 | ad 100 |
| Preservative | q.s. | q.s. |
| Dyes | q.s | q.s |
| Deodorant perfume oil | q.s | q.s |
| Neutralizing agent | q.s | q.s |
| Solubility promoter | q.s | q.s |
| Aqueous nanourea | | 10.0 |
| dispersion according | | |
| to the invention | | |
| Polyurea powder | 5.0 | |
| according to | | |
| the invention | | |

³¹Carbopol 980, Lubrizol

### 4.7 Microemulsion

| | % by wt. | % by wt. | % by wt. |
|---|---|---|---|
| | (based on | (based on | (based on |
| | the cosmetic | the cosmetic | the cosmetic |
| Raw materials | composition) | composition) | composition) |
| Glyceryl stearate | 2.0 | 2.0 | 2.0 |
| Isosteareth-20 | 4.0 | 4.0 | 4.0 |
| Octyldodecanol | 2.0 | 2.0 | 2.0 |
| Dicaprylyl carbonate | 2.0 | 2.0 | 2.0 |
| Glycerol | 3.0 | 3.0 | 3.0 |
| ACH (50% by wt. aq. | 20.0 | 20.0 | 20.0 |
| solution) | | | |
| Avocado oil | 0.10 | 0.10 | 0.10 |
| Water | ad 100 | ad 100 | ad 100 |
| Preservative | q.s. | q.s. | q.s. |
| Dyes | q.s | q.s | q.s |
| Deodorant perfume oil | q.s | q.s | q.s |
| Neutralizing agent | q.s | q.s | q.s |
| Solubility promoter | q.s | q.s | q.s |
| Aqueous nanourea | | 5.0 | 20.0 |
| dispersion according | | | |
| to the invention | | | |
| Polyurea powder | 5.0 | 5.0 | |
| according to | | | |
| the invention | | | |

### 5. Hairstyling

### 5.1 Pump-Setting Spray

| | % by wt. | % by wt. | % by wt. |
|---|---|---|---|
| | (based on | (based on | (based on |
| | the cosmetic | the cosmetic | the cosmetic |
| Raw materials | composition) | composition) | composition) |
| Acrylates copolymer³² | 2 | 2 | 2 |
| Ethanol | 55 | 55 | 55 |
| Aqueous nanourea | | 5.0 | 15.0 |
| dispersion according | | | |
| to the invention | | | |
| Polyurea powder | 5.0 | 5.0 | |
| according to | | | |
| the invention | | | |
| Perfume | q.s. | q.s. | q.s. |
| Water | ad 100 | ad 100 | ad 100 |

³²Luvimer 100 P, BASF AG

### 5.2 Aerosol Hairsprays

| | 1 | 2 | 3 | 4 | 5 |
|---|---|---|---|---|---|
| | % by wt. | | | | |
| Raw materials | (based on the cosmetic composition) | | | | |
| Aqueous nanourea dispersion | | | 10 | 5.0 | |
| according to the invention | | | | | |
| Polyurea powder according to | 5 | 10 | | 5 | 8 |
| the invention | | | | | |
| Octylacrylamide/acrylate/ | | 2.0 | | 5.0 | |
| butylaminoethyl methacrylate³³ | | | | | |
| (based on solid) | | | | | |
| Acrylates copolymer³⁴ | | | 2.0 | | 5.0 |
| Acrylates/T-butylacrylamide | 3.0 | | | | |
| copolymer³⁵ | | | | | |
| Aminomethylpropanol | | | q.s | q.s | |
| Glycerol | | 0.5 | | | |
| Panthenol | | | 0.5 | | 0.5 |
| PEG/PPG-18/18 dimethicone | | | | 0.5 | |
| PEG-12 dimethicone | | 0.05 | | | |
| Propylene glycol | | | | 0.5 | |
| Cyclomethicone | | | 1.0 | | 1.0 |
| Benzophenone-3 | | 0.1 | 0.1 | | 0.1 |
| Perfume | q.s | q.s | q.s | q.s | q.s |
| Ethanol | 14.5 | 20 | 60 | 30 | 20 |
| Water | ad | ad | ad | ad | ad |
| | 100 | 100 | 100 | 100 | 100 |
| Propane/butane 3.5 bar (20° C.) | | | 20 | 10 | |
| Dimethyl ether | 40 | 30 | | 30 | 20 |
| Fluorocarbon 152 A | | | | | 20 |

³³Amphomer, National starch
³⁴Luvimer P-100, BASF AG
³⁵Ultrahold Strong, BASF AG

### 5.3 Hair Mousse

| | |
|---|---|
| 1 | 2 |

| | |
|---|---|
| | % by wt. (based on the |
| Raw materials | cosmetic composition) |

| | | | |
|---|---|---|---|
| Aqueous nanourea | | | 10.0 |
| dispersion according | | | |
| to the invention | | | |
| Polyurea powder | 4.0 | 2.0 | |
| according to | | | |
| the invention | | | |
| Octylacrylamide/acrylate/butyl- | | 5.0 | |
| aminoethyl methacrylate³⁶ | | | |
| (based on solids) | | | |
| Acrylates copolymer³⁷ | 3.0 | | 3.0 |
| Glycerol | | 0.1 | |
| Panthenol | 0.5 | 0.05 | 0.5 |
| Polyquaternium-4 | | 2 | |
| Cetyltrimethylammonium | 0.5 | 0.2 | 0.5 |
| chloride | | | |
| PEG-12 dimethicone | 0.5 | | 0.5 |
| Cyclomethicone | 0.5 | | 0.5 |
| Benzophenone-3 | | 0.1 | |
| Perfume | q.s. | q.s. | q.s. |
| Ethanol | 10 | 15 | 10 |
| Water | ad 100 | ad 100 | ad 100 |
| Preservative | q.s. | q.s. | q.s. |
| Dimethyl ether | 7 | 10 | 7 |
| Fluorocarbon 152 A | 3 | | 3 |

³⁶Amphomer, National starch
³⁷Luvimer P-100, BASF AG

### 5.4 Hair Gel/Cream

| | 1 | 2 | 3 | 4 | 5 |
|---|---|---|---|---|---|
| Raw materials | % by wt. (based on the cosmetic composition) | | | | |
| VP/VA copolymer³⁸ | 12.0 | | | 5.0 | 5.0 |
| Acrylates copolymer³⁹ | | 4.0 | 4.0 | | |
| Aqueous nanourea | | | 10.0 | | 8.0 |
| dispersion according | | | | | |
| to the invention | | | | | |
| Polyurea powder | 5 | 2 | | 8 | 8 |
| according to the | | | | | |
| invention | | | | | |
| Carbomer | 0.8 | | | | |
| Acrylic acid/VP | | 0.5 | 0.5 | | |
| copolymer | | | | | |
| Ammonium | | | | 0.8 | 0.8 |
| acryloyldimethyl- | | | | | |
| taurate/VP copolymer | | | | | |
| Glycerol | 0.5 | | | | |
| Panthenol | | 0.5 | 0.5 | 0.5 | 0.5 |
| Propylene glycol | | | | 0.2 | 0.2 |
| Cyclomethicone | | 0.2 | 0.2 | | |
| Neutralizing agent | q.s. | q.s. | q.s. | q.s. | q.s. |
| Perfume | q.s. | q.s. | q.s. | q.s. | q.s. |
| Ethanol | 20 | | | | |
| Water | ad 100 | ad 100 | ad 100 | ad 100 | Ad 100 |
| Preservative | q.s. | q.s. | q.s. | q.s. | q.s. |

³⁸Luviskol VA 64 powder, BASF AG
³⁹Luvimer 100 P, BASF AG

### 5.6 Shampoo

| | % by wt. | % by wt. | % by wt. |
|---|---|---|---|
| | (based on | (based on | (based on |
| | the cosmetic | the cosmetic | the cosmetic |
| Raw materials | composition) | composition) | composition) |
| Sodium laureth sulphate | 7.0 | 7.0 | 7.0 |
| Cocamidopropyl betaine | 5.0 | 5.0 | 5.0 |
| Polyquaternium-10 | 0.15 | 0.15 | 0.15 |
| Diammonium lauryl | 1.5 | 1.5 | 1.5 |
| sulphosuccinate | | | |
| Water | ad 100 | ad 100 | ad 100 |
| Preservative | q.s. | q.s. | q.s. |
| Dyes | q.s | q.s | q.s |
| Perfume | q.s | q.s | q.s |
| Neutralizing agent | q.s | q.s | q.s |
| Solubility promoter | q.s | q.s | q.s |
| Aqueous nanourea | | 5.0 | 30.0 |
| dispersion according | | | |
| to the invention | | | |
| Polyurea powder | 5.0 | 5.0 | |
| according to | | | |
| the invention | | | |

### 6. Cleansing Preparations

### 6.1 Skin Cleansing Gel

| | % by wt. | % by wt. | % by wt. |
|---|---|---|---|
| | (based on | (based on | (based on |
| | the cosmetic | the cosmetic | the cosmetic |
| Raw materials | composition) | composition) | composition) |
| Sodium laureth sulphate | 13.0 | 13.0 | 13.0 |
| Cocamidopropyl betaine | 1.5 | 1.5 | 1.5 |
| Sodium cocoyl glutamate | 1.5 | 1.5 | 1.5 |
| PEG-40 hydrogenated | 0.5 | 0.5 | 0.5 |
| ricinus oil | | | |
| PEG-100 hydrogenated | 0.5 | 0.5 | 0.5 |
| glyceryl palmitate | | | |
| Polyquaternium-10 | 0.2 | 0.2 | 0.2 |
| Sodium benzoate | 0.5 | 0.5 | 0.5 |
| Sodium salicylate | 0.2 | 0.2 | 0.2 |
| Citric acid | 0.5 | 0.5 | 0.5 |
| Water | ad 100 | ad 100 | ad 100 |
| Preservative | q.s. | q.s. | q.s. |
| Dyes | q.s | q.s | q.s |
| Perfume | q.s | q.s | q.s |
| Solubility promoter | q.s | q.s | q.s |
| Aqueous nanourea | | 8.0 | 15.0 |
| dispersion according | | | |
| to the invention | | | |
| Polyurea powder | 5.0 | 8.0 | |
| according to | | | |
| the invention | | | |

### 6.2 Face Cleansing Gel

| | % by wt. | % by wt. |
|---|---|---|
| | (based on | (based on |
| | the cosmetic | the cosmetic |
| Raw materials | composition) | composition) |
| Sodium myreth sulphate | 2.0 | 2.0 |
| Decyl glucoside | 2.0 | 2.0 |
| Acrylates/C10-30 alkyl | | |
| acrylate crosspolymer⁴⁰ | 0.3 | 0.3 |
| Acrylates copolymer | 0.3 | 0.3 |
| Water | ad 100 | ad 100 |
| Neutralizing agent | q.s. | q.s. |
| Preservative | q.s. | q.s. |
| Dyes | q.s | q.s |
| Perfume | q.s | q.s |
| Solubility promoter | q.s | q.s |
| Aqueous nanourea | | 20.0 |
| dispersion according | | |
| to the invention | | |
| Polyurea powder | 7.0 | |
| according to | | |
| the invention | | |

⁴⁰Carbopol 1382, Lubrizol

### 6.3 Skin Cleansing Emulsion

| | % by wt. | % by wt. |
|---|---|---|
| | (based on | (based on |
| | the cosmetic | the cosmetic |
| Raw materials | composition) | composition) |
| Mineral oil | 10.0 | 10.0 |
| Soja oil | 10.0 | 10.0 |
| Sodium laureth sulphate | 7.5 | 7.5 |
| Sodium benzoate | 0.3 | 0.3 |
| Sodium salicylate | 0.2 | 0.2 |
| Acrylates/C10-30 alkyl | 0.8 | 0.8 |
| acrylate crosspolymer⁴¹ | | |
| Acrylates copolymer | 0.3 | 0.3 |
| Water | ad 100 | ad 100 |
| Neutralizing agent | q.s. | q.s. |
| Preservative | q.s. | q.s. |
| Dyes | q.s | q.s |
| Perfume | q.s | q.s |
| Solubility promoter | q.s | q.s |
| Aqueous nanourea | | 20.0 |
| dispersion according | | |
| to the invention | | |
| Polyurea powder | 7.0 | |
| according to | | |
| the invention | | |

⁴¹Carbopol 1382, Lubrizol

### 6.4 Shower Oil

| | % by wt. |
|---|---|
| | (based on |
| | the cosmetic |
| Raw materials | composition) |
| Helianthus annuus | ad 100 |
| Ricinus oil | 14.0 |
| MIPA laureth sulphate | 20.0 |
| Laureth-4 | 12.0 |
| Poloxamer 101 | 2.0 |
| Neutralizing agent | q.s. |
| Preservative | q.s. |
| Dyes | q.s |
| Perfume | q.s |
| Solubility promoter | q.s |
| Polyurea powder | 10.0 |
| according to | |
| the invention | |

### 6.5 Pump Foamer

| | % by wt. | % by wt. |
|---|---|---|
| | (based on | (based on |
| | the cosmetic | the cosmetic |
| Raw materials | composition) | composition) |
| Sodium cocoyl palmitate | 2.5 | 2.5 |
| Decyl glucoside | 3.0 | 3.0 |
| Polyquaternium-10 | 0.1 | 0.1 |
| PEG-200 hydrogenated | 0.5 | 0.5 |
| glyceryl palmitate | | |
| PEG-40 hydrogenated | 0.1 | 0.1 |
| ricinus oil | | |
| Sodium benzoate | 0.5 | 0.5 |
| Water | ad 100 | ad 100 |
| Preservative | q.s. | q.s. |
| Dyes | q.s | q.s |
| Perfume | q.s | q.s |
| Solubility promoter | q.s | q.s |
| Aqueous nanourea | | 20.0 |
| dispersion according | | |
| to the invention | | |
| Polyurea powder | 10.0 | |
| according to | | |
| the invention | | |

### 6.6 Oil Bath

| | % by wt. |
|---|---|
| | (based on |
| | the cosmetic |
| Raw materials | composition) |
| Laureth-2 | 10.0 |
| Mineral oil | 20.0 |
| Octyldodecanol | 20.0 |
| Isopropyl palmitate | 25.0 |
| Soya oil | ad 100 |
| Preservative | q.s. |
| Dyes | q.s |
| Perfume | q.s |
| Polyurea powder | 10.0 |
| according to | |
| the invention | |

### 6.7 Foam Bath

| | % by wt. | % by wt. | % by wt. |
|---|---|---|---|
| | (based on | (based on | (based on |
| | the cosmetic | the cosmetic | the cosmetic |
| Raw materials | composition) | composition) | composition) |
| Sodium laureth sulphate | 10.0 | 10.0 | 10.0 |
| Cocoamidopropyl betaine | 4.0 | 4.0 | 4.0 |
| Decyl glucoside | 2.5 | 2.5 | 2.5 |
| PEG-7 glyceryl cocoate | 3.0 | 3.0 | 3.0 |
| Glycerol | 5.0 | 5.0 | 5.0 |
| Sodium chloride | q.s. | q.s. | q.s. |
| Water | ad 100 | ad 100 | Ad 100 |
| Neutralizing agent | q.s. | q.s. | q.s. |
| Preservative | q.s. | q.s. | q.s. |
| Dyes | q.s | q.s | q.s |
| Perfume | q.s | q.s | q.s |
| Solubility promoter | q.s | q.s | q.s |
| Aqueous nanourea | | 10.0 | 20.0 |
| dispersion according | | | |
| to the invention | | | |
| Polyurea powder | 20.0 | 10.0 | |
| according to | | | |
| the invention | | | |

1.-13. (canceled)

14. A nanourea powder which is obtained starting from an aqueous polyurea dispersion by drying.

15. The nanourea powder according to claim 14, wherein the dispersion comprises crosslinked nanourea particles.

16. The nanourea powder according to claim 15, wherein the particles of the powder have a size of from 0.5 to 1000 μm.

17. A process for the preparation of a nanourea powder which comprises drying an aqueous dispersion of nanoureas.

18. The process according to claim 17, wherein the drying takes place with the removal of water from the dispersion at atmospheric pressure, subatmospheric pressure or superatmospheric pressure.

19. The process according to claim 17, wherein the dispersion comprises crosslinked nanourea particles.

20. The process according to claim 19, wherein the dispersion is dried by a spray-drying process.

21. A nanourea powder obtained by the process according to claim 17.

22. The nanourea powder according to claim 14 wherein the nanourea powder is incorporated into a cosmetic, coating composition, sealant, adhesive, filler, additive, auxiliary, supplement or combinations thereof.

23. A cosmetic, coating composition, sealant, adhesive, filler, additive, auxiliary, supplement or combinations thereof comprising the nanourea powder according to claim 14.

24. A cosmetic comprising an aqueous dispersion of crosslinked polyureas.

25. A process for the preparation of a cosmetic which comprises incorporating the nanourea powder according to claim 14 into the cosmetic.

26. A process for the preparation of a cosmetic which comprises incorporating an aqueous dispersion of crosslinked polyureas into the cosmetic.
